(19) United States
(12) Patent Application Publication
(10) Pub. No.: US 2025/0152674 A1
(43) Pub. Date: May 15, 2025

(54) ORAL OCTREOTIDE FOR TREATMENT OF DISEASE

(71) Applicant: AMRYT ENDO, INC., Boston, MA (US)

(72) Inventor: Asi HAVIV, Mazkeret Batya (IL)

(21) Appl. No.: 18/841,209

(22) PCT Filed: Feb. 24, 2023
(86) PCT No.: PCT/US2023/063219

§ 371 Date: Aug. 23, 2024

Related U.S. Application Data
(60) Provisional application No. 63/313,909, filed on Feb. 25, 2022.

(51) Int. Cl. A61K 38/31; A61K 9/00; A61K 9/48; A61P 1/12

(52) U.S. Cl. CPC A61K 38/31; A61K 9/0053; A61K 9/48; A61P 1/12

(57) ABSTRACT

Provided herein are methods of treating one or more symptoms associated with neuroendocrine tumors, such as diarrhea and flushing episodes associated with carcinoid tumors and methods of treating carcinoid syndrome, comprising orally administering compositions comprising octreotide or a pharmaceutically acceptable salt thereof.

### CROSS-REFERENCE TO RELATED APPLICATIONS

This application claims priority to U.S. Provisional Application No. 63/313,909 filed Feb. 25, 2022, the contents of which is herein incorporated by reference in its entirety for all purposes.

### BACKGROUND

Neuroendocrine tumors (NETs) are a heterogeneous group of cancer subtypes that arise in endocrine cells that exist in different organ systems throughout the body. Most NETs (approximately 70%) occur in the gastrointestinal (GI) tract or pancreas. Tumors arising from the GI tract are termed carcinoid tumors. NET may also occur in the respiratory tract, central nervous system, thyroid, skin, breast, and urogenital system.

Up to 20% of carcinoid tumors are estimated to have carcinoid syndrome (CS). CS are mainly associated with midgut metastatic carcinoid tumors. Most commonly, CS presents with diarrhea and flushing episodes (FEs) due to excessive secretion of serotonin; other common symptoms include abdominal pain, bronchospasm, and hypertension.

Somatostatin receptor ligands (SRLs), such as octreotide long-acting release (LAR), subcutaneous (SC) octreotide immediate release (IR), and lanreotide, are the first-line treatment for CS associated symptoms as they significantly improve FEs and diarrhea symptoms by inhibiting the secretion of serotonin among other several hormones and vasoactive substances. Current injectable SRL therapies for CS are suboptimal for patients for a variety of reasons. SRLs fail over time to maintain adequate control of CS and may follow a pattern of frequent exacerbations, specifically towards the end of the SRL injection interval, hence requiring additional medical intervention. Patients experience long-term sequelae associated with their injection, including significant persistent pain lasting for days, nodules, hemorrhage, inflammation, and scarring. Other than the physical impact there is also an emotional impact, such as frustration, anxiety, and loss of independence. Day-to-day impact is reflected by loss of work due to scheduling for injections, adverse events associated with each administration, or exacerbation of CS symptoms towards the end of the injection interval.

There is a need for an oral somatostatin receptor ligand (SRL) e.g., oral octreotide for the treatment of various diseases.

### SUMMARY

In embodiments, the present disclosure provides methods for treating diarrhea and flushing episodes associated with carcinoid tumors in a patient in need thereof, comprising orally administering a total daily dose of at least about 120 mg of octreotide or a pharmaceutically acceptable salt thereof to the patient.

In embodiments, provided herein are methods for long-term maintenance treatment of diarrhea and flushing episodes associated with a carcinoid tumor, such as a metastatic carcinoid tumor in a patient in need thereof, comprising orally administering a total daily dose of at least about 120 mg of octreotide or a pharmaceutically acceptable salt thereof to the patient.

In embodiments, the present disclosure provides methods of treating carcinoid syndrome in a patient in need thereof comprising orally administering a total daily dose of at least about 120 mg of octreotide or a pharmaceutically acceptable salt thereof to the patient.

In embodiments of the methods of the present disclosure, about 120 mg of octreotide is administered.

In embodiments of the present disclosure, the methods provided herein further comprise:
determining the patient's daily frequency of bowel movements (BMs) following administration of 120 mg of octreotide per day; and
administering about 160 mg of octreotide per day to the patient if:
   (a) the patient experiences an increased daily frequency of BMs by at least 2 BMs, compared to baseline prior to the administration of 120 mg of octreotide; and
   (b) the patient experiences≥4 BMs/day following administration of 120 mg of octreotide.

In embodiments of the present disclosure, the methods provided herein further comprise:
determining the patient's daily frequency of bowel movements (BMs) following administration of 120 mg of octreotide per day; and
administering about 160 mg of octreotide per day to the patient if:
   (a) the patient experiences an increased daily frequency of BMs by at least 2 BMs, compared to baseline prior to the administration of 120 mg of octreotide; or
   (b) the patient experiences≥4 BMs/day following administration of 120 mg of octreotide.

In embodiments of the present disclosure, the methods provided herein further comprise:
determining the patient's daily frequency of flushing episode (FE) following administration of 120 mg of octreotide per day, and
administering about 160 mg of octreotide per day to the patient if:
   (a) the patient experiences an increased daily frequency of FEs by at least 1 FE per day compared to prior to the administration of 120 mg of octreotide; or
   (b) the patient experiences an increased severity of FE following administration of 120 mg of octreotide.

In embodiments of the present disclosure, the methods provided herein further comprise:
determining the patient's carcinoid syndrome signs/symptoms following administration of 120 mg of octreotide per day; and
administering about 160 mg of octreotide per day to the patient if the patient experiences new or worsening carcinoid syndrome signs/symptoms.

In embodiments of the methods of the present disclosure, octreotide is administered in capsules, e.g., containing about 20 mg of octreotide.

In embodiments of the methods of the present disclosure, the patient treated with octreotide or a pharmaceutically acceptable salt thereof administered orally (e.g., oral octreotide capsules), has had prior treatment with a somatostatin receptor ligand (SRL) that has been shown to be effective and tolerated.

In embodiments of the methods of the present disclosure, following oral administration of octreotide or a pharmaceutically acceptable salt thereof (e.g., oral octreotide capsules), the patient experiences an improvement in diarrhea and flushing episodes associated with carcinoid tumors.

### BRIEF DESCRIPTION OF THE DRAWINGS

FIG. 1A shows geometric mean plasma octreotide concentrations (pg/mL) as a function of time (hr) (linear axes) after oral administration of single doses of oral octreotide capsules (OOC) 20 mg, 60 mg (3×20 mg capsule), and 80 mg (4×20 mg capsule) to healthy volunteers.

FIG. 1B shows geometric mean plasma octreotide concentrations (pg/mL) as a function of time (hr) (semi-logarithmic axes) after oral administration of single doses of oral octreotide capsules (OOC) 20 mg, 60 mg (3× 20 mg capsule), and 80 mg (4× 20 mg capsule) to healthy volunteers.

FIG. 2 is a schematic diagram of the study described in Example 2.

### DEFINITIONS

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . “, “about 50” means a range extending to less than half the interval(s) between the preceding and subsequent values, e.g., more than 49.5 to less than 50.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

Throughout the present disclosure, numerical ranges are provided for certain quantities. These ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “treating” as used herein with regard to a patient, refers to improving at least one symptom of the patient's disorder. Treating can be improving, or at least partially ameliorating a disorder.

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “salts” as used herein embraces pharmaceutically acceptable salts commonly used to form alkali metal salts of free acids and to form addition salts of free bases. The nature of the salt is not critical, provided that it is pharmaceutically acceptable. Suitable pharmaceutically acceptable acid addition salts can be prepared from an inorganic acid or from an organic acid. Examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

### DETAILED DESCRIPTION

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

### Neuroendocrine Tumors (NET)

There are three main types of neuroendocrine tumors, classified by origin of the tumor endocrine cells-pancreatic neuroendocrine tumors, gastrointestinal neuroendocrine tumors and pulmonary tumors. A pancreatic neuroendocrine tumor which secretes vasoactive intestinal peptide (VIP) is called VIPoma.

Neuroendocrine tumors and carcinoid syndrome and the medical treatments employed in the treatment of these conditions are described in the literature e.g., Kloppel et al 2004, Ann. N.Y. Acad Sci 1014:13-17; Strosberg (2014) Endocr. Prac. 20 (2); 167-175; Oberg et al 2004, Annals of Oncology 15:966-973; Oberg et al 2012, Annals of Oncology 23 (Supplement 7): vii124-vii130; Schmidt et al (2011) Oncogene,30:1497-1505.

Thousands of patients have neuroendocrine tumors that originate in the gastrointestinal tract and metastasize or spread to the liver or other organs. Overproduction of serotonin within these metastatic neuroendocrine tumor (mNET) cells is a driver of carcinoid syndrome, which is characterized by debilitating diarrhea, facial flushing, abdominal pain, heart valve damage, and other serious consequences. Thus, carcinoid syndrome is a subset of neuroendocrine tumors that have specific symptomatic manifestations, due to secretion of vasoactive substances into the systemic circulation.

The most common carcinoid symptoms and their related complications are summarized in the Table below.

| Symptom | Frequency | Description |
|---|---|---|
| Flushing | 90% | Subjective sensation of warmth |
| | | accompanied by reddening of the |
| | | skin anywhere on the body, |
| | | especially the face, neck, and |
| | | upper torso |
| Diarrhea | 60-80% | Secretory: intermittent accompanied |
| | | by abdominal cramping |
| | | Described as sporadic and often |
| | | accompanied by abdominal |
| | | cramping; it may become |
| | | continuous when complicated by |
| | | bacterial overgrowth |
| Mesenteric | 50% | Ischemia of vessels: decreased |
| Fibrosis | | absorption of nutrients, ascites, |
| | | intestinal obstruction, ureteral |
| | | obstruction |
| Abdominal Pain | 35% | Progressive discomfort |
| Carcinoid Heart | 19-60% | Dyspnea, holosystolic murmur |
| Disease (CHD) | | radiating to the right side of the chest |
| Bronchospasm | 15% | Wheezing |
| Pellagra | 5% | Dermatitis, diarrhea, dementia |

Most typically, CS presents with diarrhea and flushing, but other common symptoms include abdominal pain, bronchospasm, and hypertension.

The diagnosis of CS is typically suspected when a patient presents with flushing and diarrhea, regardless of whether the patient has a known NET. Symptoms are consistent throughout the clinical course of CS, and it is often the case that presentation with carcinoid symptoms facilitates a diagnosis of NET, which may be delayed due to mild and/or non-specific symptoms. Diagnosis of CS is confirmed by evidence of elevated levels of serotonin-breakdown product 5-hydroxy-indole acetic acid (5-HIAA) in the urine.

The severe and unpredictable diarrhea associated with carcinoid syndrome has a profound impact on the lives of cancer patients, often preventing them from participating in daily activities. Patients with carcinoid syndrome can live for many years with metastatic cancer, requiring the need for long-term treatment options to effectively manage their disease.

Somatostatin receptor ligand (SRL) therapy, such as Sandostatin LAR, sc Sandostatin IR, and lanreotide, remains the backbone of therapy for patients with NET, and patients are generally treated with long-acting octreotide; injectable lanreotide has also been recently used. The current standard of care for carcinoid syndrome is somatostatin analog depot injection (SSA), first approved in 1998. SSA is also termed somatostatin receptor ligand (SRL). SRL injection therapy fails over time to maintain adequate control of carcinoid syndrome for most patients, with many becoming not adequately controlled within the first two years after the therapy is initiated. This decrease in response to a drug after its administration is termed tachyphylaxis.

Current injectable SRL therapies for CS are suboptimal for patients for a variety of reasons. The injections are poorly tolerated. Patients experience long-term sequelae associated with their injection, including significant persistent pain lasting for days, nodules, hemorrhage, inflammation, and scarring. Other than the physical impact there is also an emotional impact, such as frustration, anxiety, and loss of independence. Day-to-day impact is reflected by loss of work due to scheduling for injections, adverse events associated with each administration, or exacerbation of CS symptoms towards the end of the injection interval. Direct and indirect costs of monthly injections for both patients, as well as health care providers, are therefore significant.

Long-acting SRLs may not provide a full month of somatostatin control, and some patients report a wear-off effect with recurrence of CS symptoms towards the end of the dosing interval, which sometimes necessitates supplemental short acting injections.

If metastasis of carcinoid tumor has occurred and in cases where surgical excision is not suitable, NET may be treated with currently recommended chemotherapy.

Anti-tumor agents currently used or in clinical trials to treat or palliate NET include the following: alkylating agents, doxorubicin, fluoropyrimidines e.g., 5-fluorouracil, dacarbazine, actinomycin D, platinum compounds (cisplatin, carboplatin, oxaliplatin), irinotecan, etoposide, streptozotocin (STZ), interferon alfa, interferon gamma, bortezomib (iv/sc) marketed as Velcade®, temozolomide (oral) marketed as Temodar®), bevacizumab, capecitabine and somatostatin analogs with a radioactive load (e.g., octreotide attached to a radioactive load using for example yttrium-90 or 111 indium-labeling agents; one example is Lutathera®. Lutathera (lutetium Lu 177 dotatate) is a Lu-177-labeled somatostatin analog peptide currently in development for the treatment of gastroenteropancreatic neuroendocrine tumors (GEP-NETs), including foregut, midgut, and hindgut neuroendocrine tumors in adults; see Strosberg et al (2017) New England J Med, 376:2, 125-135. Anti-tumor agents may be chemotherapeutic agents or radiotherapeutic agents. A combination of these chemotherapeutic/anti-tumor agents is typically used e.g., cisplatin/etoposide or streptozotocin/5-fluorouracil/doxorubicin or capecitabine/bevacizumab or temozolomide/capecitabine.

Other therapeutic agents which may be used to treat NET are as follows: an mTOR inhibitor such as everolimus (oral) marketed as Afinitor®), temsirolimus (intravenous) marketed as Torisel® and sirolimus (oral) marketed as Rapamune®; an oral VEGFR inhibitor such as sunitinib (marketed as Sutent®); an Src kinase inhibitor (also termed a tyrosine kinase inhibitor) e.g. bosutinib, administered orally, marketed as Bosulif®; and a tryptophan hydroxylase inhibitor e.g.telotristat etiprate also termed LX1032, administered orally. Sunitinib (sunitinib malate) is a targeted tyrosine kinase inhibitor able to inhibit members of the receptor tyrosine kinases families containing a split-kinase domain; these families include VEGF receptor (VEGFR) types 1, 2 and 3 and other receptors. Telotristat etiprate is the first investigational drug in clinical studies to target tryptophan hydroxylase, an enzyme that triggers the excess serotonin production within mNET cells that leads to carcinoid syndrome.

Chemoembolization of the hepatic artery for treatment of metastatic carcinoid tumor has been widely used in adults for treatment of NET. Other treatments, which may also be considered as required, include liver-directed therapy, such as radiofrequency ablation, radioembolization, chemoembolization, and rarely surgical debulking.

Breakthrough NET symptoms (due to hormonal symptoms associated with NETs) are common phenomena in patients receiving injectable octreotide e.g., octreotide LAR (long-acting formulation). Patients may require short-acting octreotide in addition to octreotide LAR, typically 100-250 ug up to 3 times per day for breakthrough symptoms, especially for the first 10 to 14 days after LAR injection while awaiting therapeutic levels. In patients with progressive or poorly controlled symptoms, somatostatin analog doses may be increased as needed. These additional daily s.c. injections may be effective in controlling the breakthrough symptoms, yet significantly increase the physical, emotional, and financial burden of the treatment.

In embodiments, the present disclosure provides methods for the therapy of a subject suffering from neuroendocrine tumors and carcinoid syndrome, comprising administration to the subject of a therapeutically effective amount of an oral somatostatin receptor ligand (SRL). The oral SRL may be oral octreotide, lanreotide or pasireotide or an oral formulation of DG3173; DG3173 is also termed somatoprim, and is a novel SRL, administered as subcutaneous bolus injections. The oral octreotide may be in a capsule or a tablet.

Particular advantages of the use of oral octreotide (e.g., oral octreotide capsules) e.g., administered daily as described herein, as an alternative to injectable SRLs to maintain control of CS symptoms associated with metastatic carcinoid tumors, include improved administration convenience, avoidance of often painful injections, avoidance of injection site reactions and reduction in breakthrough symptoms, improved tolerability, treatment compliance and quality of life.

### Compositions

In embodiments, octreotide or a pharmaceutically acceptable salt thereof is administered in a pharmaceutical composition comprising an oily suspension, which comprises an admixture of a hydrophobic medium and a solid form wherein the solid form comprises a therapeutically effective amount of octreotide, at least one salt of a medium chain fatty acid, and at least one matrix forming polymer. In embodiments, the matrix forming polymer is dextran or polyvinylpyrrolidone (PVP).

In embodiments, the medium chain fatty acid salt has a chain length from about 6 to about 14 carbon atoms. In embodiments, the medium chain fatty acid salt is sodium hexanoate, sodium heptanoate, sodium octanoate, sodium nonanoate, sodium decanoate, sodium undecanoate, sodium dodecanoate, sodium tridecanoate or sodium tetradecanoate, or a corresponding potassium or lithium or ammonium salt or a combination thereof. In embodiments, the medium chain fatty acid salt is a lithium, potassium or ammonium salt. In embodiments, the medium chain fatty acid salt is sodium octanoate. In embodiments, the amount of medium chain fatty acid salt in the compositions described herein may be from 10% up to about 50% by weight of the bulk pharmaceutical composition. For example, the medium chain fatty acid salt may be present at an amount about 10%-50%, or about 11%-40%, or about 11%-28% by weight, for example at about 12%-13%, 13%-14%, 14%-15%, 15%-16%, 16%-17%, 17%-18%, 18%-19%, 19%-20%, 20%-21%, 21%-22%, 22%-23%, 23%-24%, 24%-25%, 25%-26%, 26%-27%, or 27%-28%, about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% by weight of the bulk pharmaceutical composition, including any values or ranges therebetween. In specific embodiments the medium chain fatty acid salt, for example, salts of octanoic acid, salts of suberic acid, salts of geranic acid) is present at about 12%-21% by weight of the bulk pharmaceutical composition, or about 11%-18%, or about 11%-17%, 12%-16%, 12%-15%, 13%-16%, 13%-15%, 14%-16%, 14%-15%, or 15%-16%, including any values or ranges therebetween. In embodiments, the medium chain fatty acid salt is present in the pharmaceutical composition at about 15% or 16%.

In embodiments, the matrix forming polymer is polyvinylpyrrolidone (PVP). In embodiments, the PVP is present at an amount of about 2% or more by weight. In embodiments, the PVP is present in the composition at an amount of about 2% to about 20% by weight, for example, about 2%, 3%, 4%, 5%, 6%, 7%, 8%, 9%, 10%, 11%, 12%, 13%, 14%, 15%, 16%, 17%, 18%, 19%, 20%, including any values or ranges therebetween. In embodiments, the PVP is present at an amount of about 3% or more by weight. In embodiments, the PVP is present at an amount of about 3% to about 18% by weight. In embodiments, the PVP is present at an amount of about 3% to about 15% by weight. In embodiments, the PVP is present at an amount of about 5% to about 15% by weight. In embodiments, the PVP is PVP-12 and/or has a molecular weight of about 3000.

In embodiments, the hydrophobic medium comprises a mineral oil, a paraffin, a fatty acid a monoglyceride, a diglyceride, a triglyceride, an ether or an ester, or a combination thereof. In embodiments, the hydrophobic medium comprises glyceryl tricaprylate. In embodiments, the hydrophobic medium is glyceryl tricaprylate. In embodiments, the hydrophobic medium may be present at an amount about 10%-90%, by weight for example at about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90% by weight of the bulk pharmaceutical composition, including any values or ranges therebetween. In embodiments, about 20-80% of glyceryl tricaprylate by weight is present in the composition.

In embodiments, the composition further comprises a surfactant. Suitable surface-active agents include ionic and non-ionic surfactants. Examples of ionic surfactants are lecithin (phosphatidyl choline), bile salts and detergents. Examples of non-ionic surfactants include monoglycerides, cremophore, a polyethylene glycol fatty alcohol ether, a sorbitan fatty acid ester, a polyoxyethylene sorbitan fatty acid ester, Solutol HS15, or a poloxamer or a combination thereof. Examples of monoglycerides are glyceryl monocaprylate (also termed glyceryl monooctanoate), glyceryl monodecanoate, glyceryl monolaurate, glyceryl monomyristate, glyceryl monostearate, glyceryl monopalmitate, and glyceryl monooleate. Examples of sorbitan fatty acid esters include sorbitan monolaurate, sorbitan monooleate, and sorbitan monopalmitate (Span 40), or a combination thereof. Examples of polyoxyethylene sorbitan fatty acid esters include polyoxyethylene sorbitan monooleate (Tween 80), polyoxyethylene sorbitan monostearate, polyoxyethylene sorbitan monopalmitate or a combination thereof. The commercial preparations of monoglycerides that were used also contain various amounts of diglycerides and triglycerides. In embodiments, the compositions include less than about 12% by weight of total surface-active agent, for example, less than about 10%, about 9%, about 8%, about 7%, about 6%, about 5%, about 4%, about 3%, about 2%, or about 1%, including any values or ranges therebetween. In embodiments, the total sum of all the surfactants is about 6%. In embodiments, the composition comprises about 3-10% of surfactant by weight.

In embodiments, the solid consists essentially of octreotide, polyvinylpyrrolidone with a molecular weight of about 3000, and sodium octanoate. In embodiments, the composition comprises about 41% of glyceryl tricaprylate, about 27% castor oil, about 4% glyceryl monocaprylate, about 2% polyoxyethylene sorbitan monooleate, about 15% sodium octanoate, about 10% polyvinylpyrrolidone with a molecular weight of about 3000, less than 1% water and octreotide. In embodiments, the composition comprises about 68% glyceryl tricaprylate, about 4% glyceryl monocaprylate, about 2% polyoxyethylene sorbitan monooleate, about 15% sodium octanoate, about 10% polyvinylpyrrolidone with a molecular weight of about 3000, about 1% water and a therapeutically effective amount of octreotide. In embodiments, the composition comprises a therapeutically effective amount of octreotide, about 12-21% of sodium octanoate, about 5-10% of polyvinylpyrrolidone with a molecular weight of about 3000, about 20-80% of glyceryl tricaprylate, about 0-50% castor oil, and about 3-10% surfactant. In embodiments, the composition comprises a therapeutically effective amount of octreotide, about 12-21% of sodium octanoate, about 5-10% of polyvinylpyrrolidone with a molecular weight of about 3000, about 20-80% of glyceryl tricaprylate, and about 3-10% surfactant.

In embodiments, the composition comprises about 15% of sodium octanoate, about 10% of polyvinylpyrrolidone with a molecular weight of about 3000, about 30-70% glyceryl tricaprylate and about 6% of surfactant. In embodiments, the surfactant is glyceryl monocaprylate or polyoxyethylene sorbitan monooleate.

In embodiments, octreotide is present at an amount of less than 33% (e.g., less than 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.5%, or 0.1%, including any values or ranges therebetween).

In embodiments, the solid form further includes one or more excipients. In embodiments, the solid form comprises a particle or a plurality of particles. In embodiments, the solid form further comprises a stabilizer. In embodiments, the suspension comprises an admixture of a hydrophobic medium and a solid form wherein the solid form comprises a therapeutically effective amount of octreotide, at least one salt of a medium chain fatty acid and polyvinylpyrrolidone (PVP), solid form including the therapeutic agent also includes a stabilizer (e.g., a stabilizer of protein structure). Stabilizers of protein structure are compounds that stabilize protein structure under aqueous or non-aqueous conditions or can reduce or prevent aggregation of the therapeutic agent, for example during a drying process such as lyophilization or spray-drying or other processing step. Stabilizers of structure can be polyanionic molecules, such as phytic acid, polyvalent ions such as Ca, Zn or Mg, saccharides such as a disaccharide (e.g., trehalose, maltose) or an oligo or polysaccharide such as dextrin or dextran, or a sugar alcohol such as mannitol, or an amino acid such as glycine, or polycationic molecules, such as spermine, or surfactants such as Tween 80 or Span 40 or pluronic acid. Uncharged polymers, such as methyl cellulose and polyvinyl alcohol, are also suitable stabilizers.

In embodiments of the present disclosure, the dosage form comprises octreotide at an amount of about 10 mg to about 30 mg, for example, about 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, or 30 mg, including any values or ranges therebetween. In embodiments, octreotide in each dosage form is from 18 mg to about 22 mg. In embodiments, octreotide in each dosage form is about 20 mg. In embodiments, octreotide comprises octreotide acetate. In embodiments, octreotide is octreotide acetate.

In embodiments, the pharmaceutical composition includes a plurality of therapeutic agents i.e., octreotide and one (or more) additional therapeutic agents. The therapeutic agents can either be in the same solid form (e.g., in the same particle), or the therapeutic agents can each be in an independent solid form (e.g., each in different particles. In some embodiments, the therapeutic agent is in the form of a particle, for example, a granulated or solid particle. The particle is associated with or is in intimate contact with a substantially hydrophobic medium, for example, a hydrophobic medium described herein. In embodiments, the composition may include from about 1.0% to about 30% by weight of the therapeutic agent e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30% by weight. The maximum by weight of the therapeutic agent included in the composition is often in the range of about 5%-20%. In embodiments, a dosage form as described herein may be essentially a pharmaceutical product in the form in which it is marketed for use, typically involving a mixture of active drug components and nondrug components (excipients).

In embodiments, octreotide or a pharmaceutically acceptable salt thereof, is administered orally in an oral dosage form described herein. Exemplary oral dosage forms include an enteric-coated oral dosage form. Oral formulations of octreotide and methods for its use are described, for example U.S. Pat. Nos. 8,329,198, 8,535,695, 9,265,812, and the disclosures of which are incorporated by reference herein for all purposes.

In embodiments, octreotide is administered in a tablet or a capsule. In embodiments, octreotide is administered in a capsule comprising an oily suspension. In embodiments, octreotide is administered in a capsule comprising the pharmaceutical composition of the present disclosure. In embodiments, octreotide is administered in an enteric-coated. In embodiments, octreotide is administered in an enteric-coated capsule comprising 20 mg of octreotide (20 mg calculated as free base), polyvinylpyrrolidone (PVP-12), sodium caprylate, magnesium chloride, polysorbate 80, glyceryl monocaprylate, glyceryl tricaprylate, gelatin, gelatin capsules and about 6% to 7% of Acryl-EZE® (methacrylate).

### Methods of Treatment

In embodiments, the present disclosure provides methods for treating one or more symptoms in a patient with a neuroendocrine tumor, or treating a patient with a neuroendocrine tumor comprising orally administering an effective amount of octreotide or a pharmaceutically acceptable salt thereof to the patient.

In embodiments, the present disclosure provides methods for treating carcinoid syndrome, treating the symptoms of carcinoid syndrome, or treating a patient with carcinoid syndrome comprising orally administering an effective amount of octreotide or a pharmaceutically acceptable salt thereof to the patient.

In embodiments, the present disclosure provides methods for treating diarrhea and flushing episodes associated with a neuroendocrine tumor or carcinoid syndrome, comprising orally administering an effective amount of octreotide or a pharmaceutically acceptable salt thereof to the patient.

It will be understood that in embodiments of the methods provided herein, the patient is a human. In some embodiments the patient is an adult patient.

In embodiments of the methods of the present disclosure, octreotide is administered for the maintenance treatment of diarrhea and flushing episodes associated with a carcinoid tumor. In embodiments of the methods of the present disclosure, octreotide is administered for the long-term maintenance treatment of diarrhea and flushing episodes associated with a carcinoid tumor.

In embodiments of the methods of the present disclosure, the carcinoid tumor is a metastatic carcinoid tumor.

Long-term maintenance therapy in a patient suffering from the signs or symptoms of a neuroendocrine tumor (such as diarrhea and/or flushing episodes associated with a carcinoid tumor) or carcinoid syndrome continues as long as the patient is suffering from said conditions and the patient's symptoms are adequately controlled. For example, the patient's frequency of bowel movements (BMs) is maintained at less than about 4 bowel movements per day on average and/or the patient has tolerable FEs (if present). For example, the patient treated with octreotide or a pharmaceutically acceptable salt thereof administered orally (e.g., oral octreotide capsules) has Grade 1 or Grade 2, or Grade 3 FE as defined by CTCAE version 5 (v5) criteria, or a Grade 1 or Grade 2 FE as defined by CTCAE version 5 (v5) criteria. In embodiments, the patient treated with octreotide or a pharmaceutically acceptable salt thereof administered orally (e.g., oral octreotide capsules) has tolerable FEs defined as mild or moderate or not limiting instrumental activities of daily living [ADL]. In embodiments, the patient treated with octreotide or a pharmaceutically acceptable salt thereof administered orally (e.g., oral octreotide capsules) has no FEs. Thus the duration may be unlimited. In embodiments the long-term maintenance therapy may be for at least one, two, three, four or five years.

In embodiments, the methods described herein, comprise orally administering octreotide or a pharmaceutically acceptable salt thereof (e.g., oral octreotide capsules) for at least about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 24 weeks, 26 weeks, 40 weeks, 1 year or 2 years. In embodiments, the octreotide is administered for at least about 2 weeks, 4 weeks, 12 weeks, 16 weeks, 20 weeks, 26 weeks, 40 weeks, 1 year or 2 years. In embodiments, the methods described herein, comprise orally administering octreotide or a pharmaceutically acceptable salt thereof (e.g., oral octreotide capsules) for at least about 6 months, at least about 7 months, at least about 12 months, or over a duration of greater than 12 months.

In embodiments, the methods described herein, comprise orally administering octreotide or a pharmaceutically acceptable salt thereof (e.g., oral octreotide capsules) for about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 24 weeks, 25 weeks, 26 weeks, 27 weeks, 28 weeks, 29 weeks, 30 weeks, 31 weeks, 31 weeks, 32 weeks, 33 weeks, 34 weeks, 35 weeks, 36 weeks, 37 weeks, 38 weeks, 39 weeks, 40 weeks, about 1 year, about 2 years, about 3 years, about 4 years or more.

In embodiments of the present disclosure, about 5 mg to about 400 mg of octreotide is orally administered per day, including about 5 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 320 mg, 340 mg, 360 mg, 380 mg, or 400 mg of octreotide orally administered per day, including all values and subranges therebetween. In embodiments, about 10 to about 300 mg of octreotide is orally administered per day, or about 40 to about 200 mg of octreotide is orally administered per day. In embodiments, about 80-160 mg of octreotide is orally administered per day, for example twice daily. In embodiments, about 80 mg of octreotide is administered per day, wherein the dose may be administered twice per day in two doses of 40 mg each, wherein the 40 mg dose may be two 20 mg tablets. In embodiments, about 120 mg of octreotide is administered per day, wherein the dose may be administered twice per day in two doses of 60 mg of octreotide each, wherein the 60 mg dose may be three capsules comprising 20 mg of octreotide e.g., as described herein. In embodiments, about 160 mg of octreotide is administered per day, wherein the dose may be administered twice per day in two doses of 80 mg of octreotide each, wherein the 80 mg dose may be four 20 mg capsules comprising 20 mg of octreotide e.g., as described herein.

In embodiments of the present disclosure, octreotide is administered once per day, twice per day or more.

In embodiments of the present disclosure, octreotide is administered twice per day.

In embodiments of the methods of the present disclosure, orally administering octreotide or a pharmaceutically acceptable salt thereof occurs on an empty stomach e.g., at least 1 hour before a meal or at least 2 hours after a meal. In embodiments, octreotide, or a pharmaceutically acceptable salt thereof (e.g., oral octreotide capsules) is orally administered with a glass of water.

In embodiments of the methods of the present disclosure, a meal comprises 100-1000 calories, or 300-600 calories which may be a high-fat meal or a high calorie meal and may comprise carbohydrates and/or fat and or protein e.g., 100, 200, 300, 400 calories or 500-1000 calories or 700-800 calories.

In embodiments of the methods provided herein, the patient is dosed every 8-16 hours (e.g., every 12 hours). In embodiments, one administration takes place at least 6, 8, 10 or 12 hours before a second administration. In embodiments of the disclosure the first administration is in the morning (normally 5 am to noon) and the second administration is in the evening (normally 5 μm to midnight). In some embodiments of the disclosure, the administration may be self-administration; in other embodiments of the disclosure a caregiver or health professional may administer the dosage form.

In embodiments, the present disclosure provides methods for treating one or more symptoms associated with a neuroendocrine tumor in a patient in need thereof comprising orally administering a total daily dose of at least about 120 mg of octreotide or a pharmaceutically acceptable salt thereof to the patient. In embodiments, the present disclosure methods for treating carcinoid syndrome in a patient in need thereof comprising orally administering a total daily dose of at least about 120 mg of octreotide or a pharmaceutically acceptable salt thereof to the patient. In embodiments, the present disclosure provides methods for treating diarrhea and flushing episodes associated with carcinoid tumors in a patient in need thereof, comprising orally administering a total daily dose of at least about 120 mg of octreotide or a pharmaceutically acceptable salt thereof to the patient.

In embodiments of any of the methods disclosed herein, the total daily dose is about 80 mg to about 180 mg, or about 80 mg to about 160 mg, of octreotide or a pharmaceutically acceptable salt thereof. In embodiments, the total daily dose is about 120 mg of octreotide or a pharmaceutically acceptable salt thereof. In embodiments, the total daily dose is about 120 mg of octreotide or a pharmaceutically acceptable salt thereof administered twice per day (i.e., 60 mg BID).

In embodiments, if clinical symptoms (e.g., diarrhea and/or flushing episodes) of the carcinoid tumor or carcinoid syndrome are controlled (e.g., as defined herein) or response level (biochemical and symptomatic response) maintained, the patient can be maintained on 120 mg of octreotide per day. For example, in embodiments, a patient can be maintained on 120 mg of octreotide per day if the patient has an average number of daily bowel movements of less than 4 BM/day, less than 3 BM/day, less than 2 BM/day, or less than 1 BM/day and/or tolerable FEs (as defined herein e.g., by CTCAE v5 criteria).

In embodiments, the present disclosure provides method for the maintenance treatment of diarrhea and/or flushing episodes associated with metastatic carcinoid tumors in a patient in need thereof, comprising orally administering a total daily dose of at least about 120 mg of octreotide or a pharmaceutically acceptable salt thereof to the patient. In embodiments, the total daily dose is about 120 mg of octreotide or a pharmaceutically acceptable salt thereof administered twice per day (i.e., 60 mg BID).

In embodiments of the present disclosure, the patient orally administered octreotide or a pharmaceutically acceptable salt thereof (e.g., oral octreotide capsules) has had prior treatment with a somatostatin receptor ligand (SRL) for one or more signs or symptoms associated with an neuroendocrine tumor, that has been shown to be effective and tolerated. In embodiments of the present disclosure, the patient has had prior treatment for carcinoid syndrome with a somatostatin receptor ligand (SRL) that has been shown to be effective and tolerated.

In embodiments of the present disclosure, the SRL is an injectable SRL.

In embodiments of the present disclosure, the injectable SRL is octreotide, lanreotide or pasireotide. In embodiments, the injectable SRL is octreotide or lanreotide. In embodiments, the injectable octreotide is a long-acting release (LAR) octreotide formulation or a subcutaneous (SC) immediate release (IR) octreotide formulation.

In embodiments of the present disclosure, the patient was administered 10 mg, 20 mg or 30 mg of octreotide or 120 mg of lanreotide in the prior treatment.

In embodiments of the present disclosure, the patient was administered parenteral SRL monotherapy in the prior treatment, e.g., Sandostatin (octreotide) 30 mg, 20 mg, or 10 mg, or Somatuline (lanreotide) 120 mg, with a stable dose for at least 3 months of therapy at a dosing interval of 4 weeks.

In embodiments of the present disclosure, the patient was administered 120 mg of Somatuline depot (lanreotide) every 4 weeks, in the prior SRL treatment.

In embodiments of the present disclosure, the patient was administered 10 mg, 20 mg, or 30 mg of Sandostatin LAR every 4 weeks, in the prior SRL treatment.

Patients currently receiving or who have received SRL therapy by injection (e.g., as described herein) can be switched to octreotide or a pharmaceutically acceptable salt thereof given orally (e.g., oral octreotide capsules) (e.g., as described herein)—administered e.g., at an initial dose of 120 mg of octreotide per day given orally (e.g., as 60 mg BID administered for example, as three 20 mg octreotide capsules given twice per day (120 mg total)). If clinical symptoms are controlled or response level (biochemical and symptomatic response maintained), the patient can be maintained on 120 mg of octreotide per day.

In case of symptomatic exacerbation or breakthrough symptoms the dose may be adjusted e.g., from 120 mg of octreotide per day given orally to 160 mg of octreotide per day given orally (e.g., administered 80 mg BID, for example, as four 20 mg octreotide capsules administered twice per day (160 mg total)) as described herein. Once clinical symptoms are controlled, the dose can be further adjusted to 120 mg of octreotide per day.

In case of tolerability issues (e.g., severe nausea or headache assessed as related to octreotide), the dose may be temporarily adjusted from 120 mg of octreotide per day to 80 mg of octreotide per day (e.g., administered 40 mg BID for example, as two 20 mg octreotide capsules administered twice per day (80 mg total) as described herein) given orally.

In embodiments of the methods of the present disclosure, the method further comprises:
determining the patient's daily frequency of bowel movements (BMs) following administration of 120 mg of octreotide per day; and
administering about 160 mg of octreotide per day to the patient if:
   (a) the patient experiences an increased daily frequency of BMs by at least 2 BMs, compared to baseline prior to the administration of 120 mg of octreotide; and
   (b) the patient experiences≥4 BMs/day following administration of 120 mg of octreotide.

In embodiments, 160 mg of octreotide is administered if the patient (a) experiences an increased daily frequency of BMs by 1 BM, 2 BMs, 3 BMs, 4 BMs, 5 BMs, 6 BMs, or more, including any values or ranges therebetween. In embodiments, 160 mg of octreotide is administered if the patient (b) experiences 4 BMs, 5 BMs, 6 BMs, 7 BMs, 8 BMs, 9 BMs, 10 BMs, 11 BMs, 12 BMs or more following administration of 120 mg of octreotide, including any values or ranges therebetween.

In embodiments of the methods of the present disclosure, the method further comprises:
determining the patient's daily frequency of flushing episode (FE) following administration of 120 mg of octreotide per day, and administering about 160 mg of octreotide per day to the patient if:
   (a) the patient experiences an increased daily frequency of FEs by at least 1 FE per day compared to prior to the administration of 120 mg of octreotide; or
   (b) the patient experiences an increased severity of FE (e.g., as determined by an eDiary or based on CTCAE v5 grading) following administration of 120 mg of octreotide.

In embodiments, 160 mg of octreotide is administered if the patient (a) experiences an increased daily frequency of FEs by 1 FE, 2 FEs, 3 FEs, 4 FEs, 5 FEs, 6 FEs, or more, including any values or ranges therebetween, following administration of 120 mg of octreotide.

In embodiments of the methods of the present disclosure, the method further comprises:
determining the patient's daily frequency of flushing episode (FE) following administration of 120 mg of octreotide per day, and
administering about 160 mg of octreotide per day to the patient if:
the patient experiences an increased severity of FE (e.g., as determined by an eDiary or based on CTCAE v5 grading) following administration of 120 mg of octreotide.

In embodiments of the methods disclosed herein, the patient experiences an increased severity of FE as determined by CTCAE v5 grading.

In embodiments of the methods disclosed herein, increased severity of FE is a grade 2 for at least 7 of prior 14 days or grade 3 based on CTCAE v5 grading.

In embodiments of the methods of the present disclosure, the method further comprises:
determining the patient's carcinoid syndrome signs/symptoms following administration of 120 mg of octreotide per day; and
administering about 160 mg of octreotide per day to the patient if the patient experiences new or worsening carcinoid syndrome signs/symptoms.

In embodiments of the present disclosure, 160 mg of octreotide is administered per day for about 2 days to about 40 days, including about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 32 days, about 33 days, about 34 days, about 35 days, about 36 days, about 37 days, about 38 days, about 39 days, about 40 days, including all ranges and values therebetween.

In embodiments of the present disclosure, 160 mg of octreotide is administered per day for about 2 days to about 14 days. In embodiments of the present disclosure, 160 mg of octreotide is administered per day for about 4 days to about 7 days.

In embodiments of the methods of the present disclosure, the method further comprises:
determining the patient's carcinoid syndrome signs/symptoms following administration of 160 mg of octreotide per day; and
administering about 120 mg of octreotide per day to the patient if the patient's biochemical and/or symptomatic response is controlled.

In embodiments of the methods of the present disclosure, the method further comprises:

further comprising:
determining the patient's tolerability following administration of 120 mg of octreotide per day; and
administering about 80 mg of octreotide per day to the patient if the patient experiences tolerability issues following administration of 120 mg of octreotide per day.

In embodiments, the present disclosure provides methods to prevent or treat breakthrough symptoms (e.g., related to carcinoid syndrome such as diarrhea, flushing episodes or abdominal pain occur) comprising orally administering octreotide or a pharmaceutically acceptable salt thereof (e.g., oral octreotide capsules). In embodiments, at least about 120-160 mg of octreotide is administered orally per day to prevent or treat breakthrough symptoms.

In embodiments of the present disclosure, octreotide is administered in capsules. In embodiments, each capsule contains 20 mg of octreotide. In embodiments of the present disclosure, the capsule is enterically coated. In embodiments, the pharmaceutically acceptable salt is octreotide acetate.

In embodiments of the present disclosure, octreotide is administered in a capsule comprising an oily suspension (e.g., as described herein).

In embodiments of the present disclosure, the oily suspension comprises an admixture of a hydrophobic medium and a solid form, wherein the solid form comprises octreotide and at least one salt of a medium chain fatty acid.

In embodiments of the present disclosure, the oily suspension comprises an admixture of a hydrophobic medium and a solid form, wherein the solid form comprises octreotide, at least one salt of a medium chain fatty acid, and polyvinylpyrrolidone (PVP), wherein the at least one salt of a medium chain fatty acid is present in the dosage form at an amount of at least 12% by weight and wherein the PVP is present in the dosage form at an amount of 3% or more by weight.

In embodiments of the present disclosure, the composition comprises octreotide, about 12-21% of sodium octanoate by weight, about 5-15% of polyvinylpyrrolidone by weight, about 20-80% of glyceryl tricaprylate by weight, and about 3-10% of surfactant by weight.

In embodiments, particular advantages of oral administration of octreotide are avoidance of often painful injections, avoidance of injection site reactions and reduction in breakthrough symptoms. Other advantages of daily treatment with octreotide or a pharmaceutically acceptable salt thereof given orally (e.g., oral octreotide capsules), may include for example, no pharmacodynamic wear-off effects at the end of the administration interval (as noted in many patients during the last week of the SRLs injection interval).

In embodiments of the present disclosure after treatment with octreotide or a pharmaceutically acceptable salt thereof administered orally (e.g., oral octreotide capsules), the patient experiences an improvement in diarrhea and the severity flushing episodes associated with carcinoid tumors.

In embodiments of the present disclosure, after treatment with octreotide or a pharmaceutically acceptable salt thereof administered orally (e.g., oral octreotide capsules), the patient experiences an improvement in diarrhea.

In embodiments of the present disclosure, after treatment with octreotide or a pharmaceutically acceptable salt thereof administered orally (e.g., oral octreotide capsules), the patient experiences an improvement in the severity flushing episodes associated with carcinoid tumors as determined by CTCAE v5 grading.

The CTCAE v5 grading displays Grades 1 through 5 with unique clinical descriptions of severity for each adverse event (AE) based on this general guideline:
Grade 1 Mild; asymptomatic or mild symptoms; clinical or diagnostic observations only; intervention not indicated.
Grade 2 Moderate; minimal, local or noninvasive intervention indicated (e.g., short course of antibiotics); limiting age-appropriate instrumental Activities of Daily Living (ADL).
Grade 3 Severe or medically significant but not immediately life-threatening; hospitalization or prolongation of hospitalization indicated; disabling; limiting self-care ADL.
Grade 4 Life-threatening consequences; urgent intervention indicated. In embodiments, Life-threatening event refers to an event in which the subject was at risk of death at the time of the event, e.g., as judged by the treating physician; urgent/emergent intervention indicated.
Grade 5 Death related to AE.

In embodiments of the present disclosure after treatment with octreotide or a pharmaceutically acceptable salt thereof administered orally (e.g., oral octreotide capsules), the patient's symptoms are adequately controlled. For example, the patient has a normal frequency of bowel movements (BMs)<4.0/day on average and/or tolerable FEs (e.g., defined as Grade 1, 2, or 3 based on CTCAE v5 criteria; or Grade 1 or 2 based on CTCAE v5 criteria. In embodiments, tolerable FEs are defined as asymptomatic or moderate and/or not limiting instrumental activities of daily living [ADL], or defined as mild or moderate and/or not limiting activities of daily living [ADL]).

In embodiments, the patient orally administered octreotide or a pharmaceutically acceptable salt thereof (e.g., oral octreotide capsules) has Grade 1 or 2 FEs (if present) based on CTCAE v5 criteria, for example, asymptomatic or moderate symptoms, or limiting instrumental activities of daily living (ADL)).

In embodiments, the patient orally administered octreotide or a pharmaceutically acceptable salt thereof (e.g., oral octreotide capsules) has Grade 1 or 2 FEs (if present) based on CTCAE v5 criteria, for example, asymptomatic or moderate symptoms, or limiting instrumental activities of daily living (ADL)).

In embodiments, the patient orally administered octreotide or a pharmaceutically acceptable salt thereof (e.g., oral octreotide capsules) has Grade 3 FEs (if present) based on CTCAE v5 criteria for example, associated with hypotension and/or tachycardia or limiting self-care ADL.

In embodiments, the patient orally administered octreotide or a pharmaceutically acceptable salt thereof (e.g., oral octreotide capsules) has no flushing episodes, or Grade 1 or 2 FEs (if present) based on CTCAE v5 criteria. In embodiments, the patient orally administered octreotide or a pharmaceutically acceptable salt thereof (e.g., oral octreotide capsules) has no flushing episodes, or Grade 1, 2 or 3 FEs (if present) based on CTCAE v5 criteria.

In embodiments of the present disclosure after treatment with octreotide or a pharmaceutically acceptable salt thereof administered orally (e.g., oral octreotide capsules), the patient has an average number of daily bowl movements of less than about 4, less than about 3, less than about 2, or less than about 1. In embodiments, after treatment the patient has an average number of daily bowl movements of 0-4, 0-3, 0-2, 0-1, 1-4, 1-3, 1-2, or 2-4 BM/day.

In embodiments, after at least about 1-20 weeks of treatment with octreotide or a pharmaceutically acceptable salt thereof administered orally (e.g., oral octreotide capsules), the patient has an average number of daily bowl movements of less than about 4, less than about 3, less than about 2, or less than about 1.

In embodiments, after treatment with octreotide or a pharmaceutically acceptable salt thereof administered orally (e.g., oral octreotide capsules) for about 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 52 weeks, 79 weeks, 104 weeks or more, the patient has an average number of daily bowl movements of less than about 4, less than about 3, less than about 2, or less than about 1. In embodiments, the patient has an average number of daily bowl movements of less than about 4, less than about 3, less than about 2, or less than about 1, during the prior 4 weeks.

In embodiments, after about 3-20 weeks, or 4-16 weeks of treatment with octreotide or a pharmaceutically acceptable salt thereof administered orally (e.g., oral octreotide capsules) including about from about 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, about 17 weeks, about 18 weeks, about 19 weeks, to about 20 weeks, including all values and ranges therebetween, the patient has an average number of daily bowl movements of less than about 4, less than about 3, less than about 2, or less than about 1. In embodiments, the patient has an average number of daily bowl movements of less than about 4, less than about 3, less than about 2, or less than about 1, during the prior 4 weeks.

In embodiments of the present disclosure after about 16 weeks of treatment with octreotide or a pharmaceutically acceptable salt thereof administered orally (e.g., oral octreotide capsules) the patient has an average number of daily bowl movements of less than about 4 during the last 4 weeks (i.e., week 13-16).

In embodiments of the present disclosure after treatment with octreotide or a pharmaceutically acceptable salt thereof administered orally (e.g., oral octreotide capsules) e.g., as disclosed herein, the patient has tolerable FEs (defined for example, by CTCAE version 5 (v5) criteria e.g., as Grade 1, Grade 2, or Grade 3 FE by CTCAE version 5 (v5) criteria, or a Grade 1 or Grade 2 FE by CTCAE version 5 (v5) criteria). In embodiments, after treatment with octreotide or a pharmaceutically acceptable salt thereof administered orally (e.g., oral octreotide capsules) as disclosed herein, for about 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 20 weeks, 25 weeks, 52 weeks, 79 weeks, 104 weeks or more the patient has tolerable FEs (defined for example, by CTCAE version 5 (v5) criteria e.g., as Grade 1, Grade 2, or Grade 3 FE by CTCAE version 5 (v5) criteria, or a Grade 1 or Grade 2 FE by CTCAE version 5 (v5) criteria). In embodiments, after about 4-16 weeks of treatment the patient has tolerable FEs (defined for example, by CTCAE version 5 (v5) criteria e.g., as Grade 1, Grade 2, or Grade 3 FE by CTCAE version 5 (v5) criteria, or a Grade 1 or Grade 2 FE by CTCAE version 5 (v5) criteria).

In embodiments of the present disclosure after treatment with octreotide or a pharmaceutically acceptable salt thereof administered orally (e.g., oral octreotide capsules), the patient has an average number of bowel movements per day (BM/day) that has not increased by more than 1.0 BM/day compared to baseline prior to treatment, or compared to prior treatment with injectable SRL (e.g., as disclosed herein).

In embodiments of the present disclosure after treatment with octreotide or a pharmaceutically acceptable salt thereof administered orally (e.g., oral octreotide capsules) as disclosed herein, there is a reduction in the average number of daily flushing episodes to an average daily flushing episodes of about 1 to 4 episode/day, about 1 to 3 episode/day, or about 1 to 2 episode/day, or about 0.5 to 1 episode/day.

To assess patient perception of the observed changes in BM frequency and/or severity/frequency of FE that are meaningful for the patients treated by methods disclosed herein, a Patient Perception Questionnaire (PPQ) can be utilized. In embodiments, patients may complete a PPQ to address their perception of change in BM and FE (3 separate questions), on orally administered octreotide (e.g., oral octreotide capsules) compared to SRL injections (prior to treatment with oral octreotide), (“much better”, “a little better”, “the same”, “a little worse”, “much worse”), and their overall satisfaction with orally administered octreotide (e.g., oral octreotide capsules) compared to SRL injections in control of their BM and FE (additional 2 questions) (“very satisfied”, “satisfied”, “neither satisfied nor dissatisfied”, “dissatisfied”, “very dissatisfied”).

In embodiments, after treatment with octreotide or a pharmaceutically acceptable salt thereof administered orally (e.g., oral octreotide capsules) e.g., as described herein the patient experiences an improvement in BM frequency and/or in severity/frequency of FE compared to SRL injections (prior to treatment with oral octreotide) as assessed by a Patient Perception Questionnaire (PPQ). For example, in embodiments, after treatment with octreotide or a pharmaceutically acceptable salt thereof administered orally (e.g., oral octreotide capsules) the patient experiences an improvement in BM frequency and/or in severity/frequency of FE characterized BM frequency and/or in severity/frequency of FE characterized as “the same”, “a little better” or “much better” as assessed by a PPQ compared to SRL injections (prior to treatment with oral octreotide). In embodiments, the patient experiences an improvement in BM frequency and/or in severity/frequency of FE characterized as “a little better” or “much better” as assessed by a PPQ compared to SRL injections (prior to treatment with oral octreotide). In embodiments, the patient experiences an improvement in BM frequency and/or in severity/frequency of FE characterized as “much better” as assessed by a PPQ compared to SRL injections (prior to treatment with oral octreotide).

To assess the presence of symptoms like diarrhea and associated functional limitations (i.e., embarrassment, urgency, limiting activities, fears of being far from the toilet) in patients treated by methods disclosed herein a Functional Assessment of Cancer Therapy-Carcinoid Syndrome Symptom Index (FACT-CSI) questionnaire can be utilized. (Shaunfield, S., Webster, K. A., Kaiser, K., Greene, G. J., Yount, S. E., Lacson, L., et al. (2020). “Development of the Functional Assessment of Cancer Therapy-Carcinoid Syndrome Symptom Index (FACT-CSI).” Neuroendocrinology). The FACT-CSI questionnaires contain 20 items. Each item is graded on a 0 to 4 point scale.

In embodiments of the methods of the present disclosure, Response Evaluation Criteria in Solid Tumors (RECIST) v1.1 and radiographic imaging can be used to assess progression free survival and objective tumor response rate in the methods disclosed herein.

In embodiments, radiographic imaging (CT or MRI) is performed about every 3 months.

In embodiments of the methods disclosed herein, after treatment with octreotide or a pharmaceutically acceptable salt thereof administered orally (e.g., oral octreotide capsules) the patient experiences an improvement in progression-free survival of the subject suffering from a NET (such as a carcinoid tumor) or carcinoid syndrome.

In embodiments of the methods of the present disclosure, a Carcinoid Syndrome-Treatment Satisfaction Questionnaire (CS-TSQ) can be utilized to assess treatment satisfaction for adult subjects with CS symptoms treated with oral octreotide (e.g., compared to baseline prior to treatment, or compared to SRL injections (prior to treatment with oral octreotide).

The CS-TSQ is focused on the following aspects:
CS Symptoms
Injection Site Reactions
Treatment Administration Bother
Treatment Convenience
Emotional Impact
Treatment Satisfaction

In embodiments, after treatment with octreotide or a pharmaceutically acceptable salt thereof administered orally (e.g., oral octreotide capsules) the patient experiences an improvement in treatment satisfaction as assessed by CS-TSQ compared to baseline prior to treatment.

In embodiments, after treatment with octreotide or a pharmaceutically acceptable salt thereof administered orally (e.g., oral octreotide capsules) e.g., as described herein the patient experiences an improvement in treatment satisfaction as assessed by CS-TSQ compared to SRL injections (prior to treatment with oral octreotide).

In some embodiments, the present methods provide an AUC_{0-inf} (expressed in terms of hr*pg/mL) levels of octreotide that correlate to one or more statistically significant therapeutic effects. In some embodiments, the therapeutically effective AUC_{0-inf} levels of octreotide provided by the methods of the present disclosure range from about 5,000 hr*pg/mL to about 500,000 hr*pg/mL, including about 5,000 hr*pg/mL, about 10,000 hr*pg/mL, about 15,000 hr*pg/mL, about 20,000 hr*pg/mL, about 25,000 hr*pg/mL, about 30,000 hr*pg/mL, about 35,000 hr*pg/mL, about 40,000 hr*pg/mL, about 45,000 hr*pg/mL, about 50,000 hr*pg/mL, about 55,000 hr*pg/mL, about 60,000 hr*pg/mL, about 65,000 hr*pg/mL, about 70,000 hr*pg/mL, about 75,000 hr*pg/mL, about 80,000 hr*pg/mL, about 85,000 hr*pg/mL, about 90,000 hr*pg/mL, about 100,000 hr*pg/mL, about 110,000 hr*pg/mL, about 120,000 hr*pg/mL, about 125,000 hr*pg/mL, about 150,000 hr*pg/mL, about 200,000 hr*pg/mL, about 250,000 hr*pg/mL, about 300.00 hr*pg/mL, about 350,000 hr*pg/mL, about 400,000 hr*pg/mL, about 450 hr*pg/mL, to about 500,000 hr*pg/mL, including all ranges and values therebetween. In embodiments, the therapeutically effective AUC_{0-inf} levels of octreotide provided by the methods of the present disclosure range from about 15,000 hr*pg/mL to about 350,000 hr*pg/mL, about 15,000 hr*pg/mL to about 50,000 hr*pg/mL, or about 25,000 hr*pg/mL to about 35,000 hr*pg/mL, or about 35,000 hr*pg/mL to about 45,000 hr*pg/mL. In embodiments, the therapeutically effective AUC_{0-inf} levels of octreotide is provided by administering a dose of octreotide or a pharmaceutically acceptable salt thereof which is about 40 mg. In embodiments, the therapeutically effective AUC_{0-inf} levels of octreotide is provided by administering a dose of octreotide or a pharmaceutically acceptable salt thereof which is about 60 mg. In embodiments, the therapeutically effective AUC_{0-inf} levels of octreotide is provided by administering a dose of octreotide or a pharmaceutically acceptable salt thereof which is about 80 mg. In embodiments, the therapeutically effective AUC_{0-inf} levels of octreotide is provided by administering a dose of octreotide or a pharmaceutically acceptable salt thereof which is about 120 mg. In embodiments, the therapeutically effective AUC_{0-inf} levels of octreotide is provided by administering a dose of octreotide or a pharmaceutically acceptable salt thereof which is about 160 mg.

In some embodiments, the present methods provide a Cmax (expressed in terms of pg/mL) levels of octreotide that correlate to one or more statistically significant therapeutic effects. In some embodiments, the therapeutically effective Cmax levels of octreotide provided by the methods of the present disclosure range from about 100 μg/mL to about 100,000 μg/mL, including about 100 μg/mL, about 500 μg/mL, about 1000 μg/mL, about 2,000 μg/mL, about 2500 μg/mL, about 3,000 μg/mL, about 4,000 μg/mL, about 4,500 μg/mL, about 4,600 μg/mL, about 4,700 μg/mL, about 4,800 μg/mL, about 5,000 μg/mL, about 5,500 μg/mL, about 6,000 μg/mL, about 6,500 μg/mL, about 7,000 μg/mL, about 7,500 μg/mL, about 8,000 μg/mL, about 8,500 μg/mL, about 9,000 μg/mL, about 9,500 μg/mL, about 10,000 μg/mL, about 11,000 μg/mL, about 12,000 μg/mL, about 13,000 μg/mL, about 15,000 μg/mL, about 20,000 μg/mL, about 30,000 μg/mL, about 40,000 μg/mL, about 50,000 μg/mL, about 60,000 μg/mL, about 70,000 μg/mL, about 80,000 μg/mL, about 90,000 μg/mL, to about 100,000 μg/mL, including all ranges and values therebetween. In embodiments, the therapeutically effective Cmax of octreotide is provided by administering a dose of octreotide or a pharmaceutically acceptable salt thereof which is about 40 mg. In embodiments, the therapeutically effective Cmax of octreotide is provided by administering a dose of octreotide or a pharmaceutically acceptable salt thereof which is about 60 mg. In embodiments, the therapeutically effective Cmax of octreotide is provided by administering a dose of octreotide or a pharmaceutically acceptable salt thereof which is about 80 mg. In embodiments, the therapeutically effective Cmax of octreotide is provided by administering a dose of octreotide or a pharmaceutically acceptable salt thereof which is about 120 mg. In embodiments, the therapeutically effective Cmax of octreotide is provided by administering a dose of octreotide or a pharmaceutically acceptable salt thereof which is about 160 mg.

### EXAMPLES

### Example 1: Phase 1 PK Studies

This study is an open-label, randomized, 6-sequence by 3-period crossover study. The primary objective of the study was to evaluate the pharmacokinetic (PK) profile of oral octreotide 20 mg (administered as 1× 20 mg capsule), 60 mg (administered as 3× 20 mg capsules), and 80 mg (administered as 4× 20 mg capsules) in 30 healthy volunteers.

### Treatment

Subjects will receive each of the following treatments separated by a 48-hour washout period:
Treatment A: oral octreotide capsules 1×20 mg (20 mg)
Treatment B: oral octreotide capsules 3×20 mg (60 mg)
Treatment C: oral octreotide capsules 4×20 mg (80 mg)

To maintain a balanced design with 3 treatments, a Williams design with 6 sequences and 3 periods will be used. The sequences are as follows: ABC, BCA, CAB, CBA, ACB, and BAC. Subjects will be randomly assigned to 1 of the 6 sequences.

### Screening Phase

Eligibility will be determined during the Screening Phase.

### Treatment Phase

For each treatment period, after an overnight fast of at least 10 hours, subjects will receive a single dose of 1 of the 3 treatments (on Days 1, 3 and 5). Subjects will continue to fast for 4 hours post dose. Afterward, subjects will be served a standardized lunch, dinner, and evening snack.

During each treatment period, blood samples for determination of octreotide plasma concentrations will be collected over a 24-hour period following each dose (from the time the last capsule is consumed) at the following time points: pre dose (within 15 minutes prior to dosing), 0.083 (5 minutes), 0.167 (10 minutes), 0.33 (20 minutes), 0.5 (30 minutes), 0.67 (40 minutes), 0.75 (45 minutes), 1, 1.33, 1.67, 2, 2.33, 2.67, 3, 3.33, 3.67, 4, 4.5, 5, 6, 8, 10, 12, 15, and 24 hours post dose.

Plasma concentrations of octreotide will be determined by means of a validated liquid chromatography with tandem mass spectrometry (LC-MS/MS) assay.

The following PK parameter estimates will be calculated by noncompartmental methods for each treatment:

“Area under the concentration-time curve (AUC) from time 0 to the last measurable concentration (AUC₀₋ₗₐₛₜ)
AUC from time 0 extrapolated to infinity (AUC_{0-inf})
Peak concentration observed (Cₘₐₓ)”
Time to peak concentration (Tₘₐₓ)
Absorption lag time (T_{lag})
Elimination rate constant (λz) and half-life (t½)

Additional PK parameter estimates will be calculated if deemed necessary.

### Safety

Safety will be evaluated descriptively based on the following assessments: adverse events (AEs, including serious AEs [SAEs]); vital signs (blood pressure, heart rate, respiration rate, and body temperature); 12-lead ECGs; physical examinations; safety laboratory evaluations

All doses will be removed from blisters and administered one by one, separated by a 30 second gap between each capsule in treatments B and C.

Subjects will be confined in the clinic from check-in on Day-1 until discharge on Day 6.

### Follow-Up Phase

Approximately 7 days following completion of the last treatment period (i.e., administration of the last test condition), subjects will return to the clinic for a follow-up EOS/ET visit to undergo safety assessments.

### Results

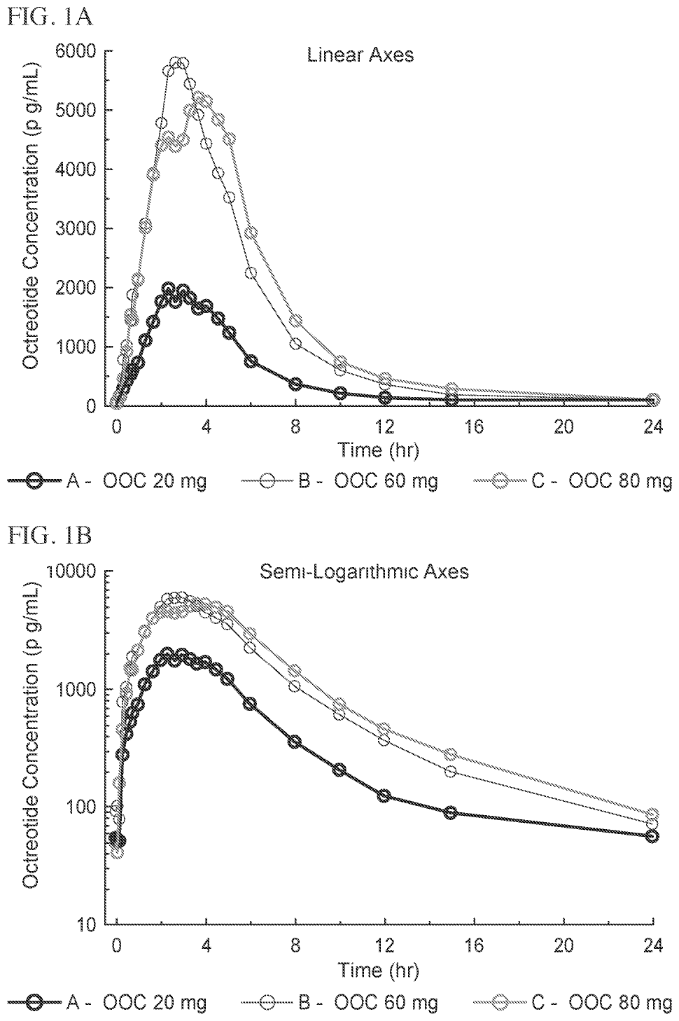

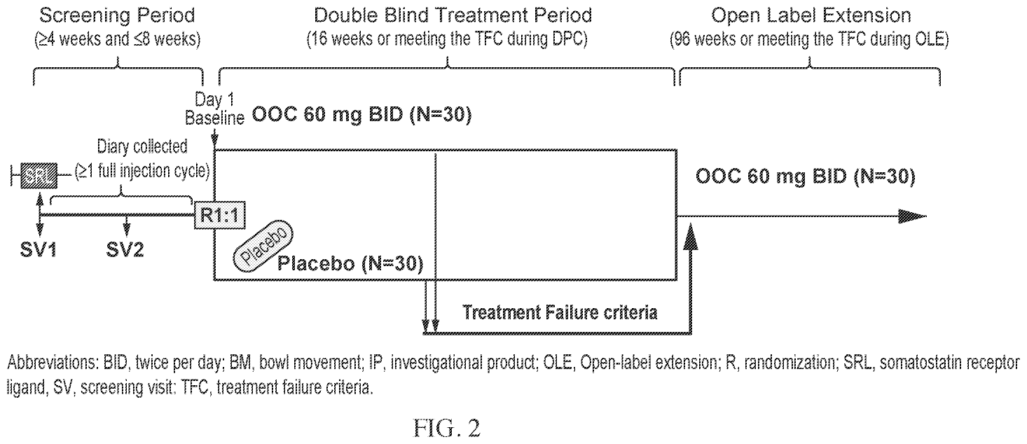

FIG. 1A and FIG. 2A show the geometric mean plasma octreotide concentrations-linear (FIG. 1A) and semi-logarithmic (FIG. 2A) axes-after oral administration of single doses of oral octreotide capsule (OOC) 20 mg, 60 mg, and 80 mg to healthy volunteers.

TABLE 1A
PK results for all subjects

| | Treatment A | Treatment B | Treatment C |
|---|---|---|---|
| Parameter* | OOC 20 mg | OOC 60 mg | OOC 80 mg |
| Tlag (hr) | 0.50 (25) | 0.33 (26) | 0.33 (26) |
| | [0.17-3.67] | [0.17-1.00] | [0.17-2.67] |
| Cmax (pg/mL) | 3,193 [137] (28) | 8,774 [127] (28) | 8,907 [163] (27) |
| Tmax (hr) | 2.33 (28) | 2.67 (28) | 2.67 (27) |
| | [0.5-5.00] | [0.33-5.00] | [0.33-6.00] |
| AUC(0-t) | 10,368 [133] (28) | 32,765 [95.9] (28) | 35,980 [149] (27) |
| (hr*pg/mL) | 10,682 [130] (28) | 33,181 [95.5] (28) | 41,297 [148] (24) |
| AUC(inf) | 0.2005 [22.4] (28) | 0.1942 [23.7] (28) | 0.1795 [22.3] (24) |
| (hr*pg/mL) | 3.46 [22.4] (28) | 3.57 [23.7] (28) | 3.86 [22.3] (24) |
| λz (1/hr) | | | |
| t1/2 (hr) | | | |

*geometric mean [geometric CV %] (N) except Tlag and Tmax for which the median (N) [Range] are reported

As shown in Table 1A, 80 mg oral octreotide provides exposure 3.9 times higher than 20 mg oral octreotide (geometric mean ratios of AUC (inf)) and 1.24 times higher than 60 mg, oral octreotide.

The 60 mg provides exposure 3.1 times higher than 20 mg.

Safety and tolerability was acceptable. No serious adverse events were reported.

TABLE 1B
PK results excluding subject #02

| | Treatment A | Treatment B | Treatment C |
|---|---|---|---|
| Parameter* | OOC 20 mg | OOC 60 mg | OOC 80 mg |
| Tlag (hr) | 0.50 (25) | 0.33 (26) | 0.33 (25) |
| | [0.17-3.67] | [0.17-1.00] | [0.33-2.67] |
| Cmax (pg/mL) | 2,816 [93.9] (27) | 8,131 [113] (27) | 7,931 [128] (26) |
| Tmax (hr) | 2.33 (27) | 2.67 (27) | 2.83 (26) |
| | [0.5-5.00] | [0.33-5.00] | [0.33-6.00] |
| AUC(0-t) | 9,150 [91.0] (27) | 30,015 [75.8] (27) | 32,443 [123] (26) |
| (hr*pg/mL) | 9,432 [88.2] (27) | 30,384 [75.2] (27) | 36,933 [120] (23) |
| AUC(inf) | 0.2018 [22.6] (27) | 0.1955 [23.9] (27) | 0.1797 [22.8] (23) |
| (hr*pg/mL) | 3.43 [22.6] (27) | 3.55 [23.9] (27) | 3.86 [22.8] (23) |
| λz (1/hr) | | | |
| t1/2 (hr) | | | |

*geometric mean [geometric CV%] (N) except Tlag and Tmax for which the median (N) [Range] are reported

As shown in Table 1B the concentrations measured for subject #02 were much higher than for any other subject. In a Grubb's test on Cₘₐₓ for the 3 treatments subject #02 was a significant outlier (p<0.05). When excluded, the variability (overall between subject geometric CVs) is lower for Cₘₐₓ and AUC. However, the subject is consistent across the 3 treatments—high for all compared to the others, suggesting this subject may handle octreotide differently. Since subject #02 was high for all 3 treatments the subject was included in the analysis (Table 1).

### Example 2: Clinical Study

This study is a Phase 3, randomized, double-blind, placebo-controlled, multicenter study to evaluate efficacy and safety of oral octreotide capsules (OOC) in subjects with documented history of carcinoid syndrome (diarrhea, with or without flushing episodes (FEs)) associated with stable well-differentiated metastatic carcinoid tumors who are treated with SRL injections (octreotide or lanreotide) with a stable dose for at least 3 months and their symptoms are adequately controlled.

The Core study will consist of 2 periods: a screening period followed by a double-blind Placebo-controlled (DPC) period. The DPC Core study is a Phase 3, randomized, double-blind, placebo-controlled, multicenter, 16-week study evaluating efficacy, PK, safety and subject reported outcomes of OOC.

The Core study will be followed by an open-label extension (OLE), multicenter, ≥48 weeks study evaluating long-term safety and tolerability and progression free survival of OOC.

A schematic of the study design is presented in FIG. 2, and the study duration is shown in the table below.

| | |
|---|---|
| Screening period: | ≥4 weeks and up to 8 weeks |
| DPC Treatment period: | 16 weeks or earlier if meeting |
| | the TFC during DPC |
| OLE Treatment period | 96 weeks or earlier if meeting |
| | the TFC during OLE |
| Follow-up period | 4 weeks from last dose of IP |

### Screening Period (4 to 8 Weeks Prior to Baseline)

After signing informed consent, subjects will be screened for study eligibility.

The screening period will consist of 2 in-clinic screening visits, screening visit 1 (SV1) should occur at or within 3 days after the last SRL injection and screening visit 2 (SV2). SV2 should be scheduled at the beginning of the 3^{rd} week of the SRL injection interval (−3 days). The screening period is expected to last≥4 weeks and up to 8 weeks.

During the screening period daily eDiary of BMs and FEs will be collected, as well as use of anti-diarrheals and/or Sandostatin SC. Eligibility will be determined based on the BMs and FEs during the ˜4 weeks of the SRL injection interval prior to baseline.

Collection of plasma 5-HIAA and a single time point octreotide PK assessment will be done at SV1 and SV2. Collection of 24-hour urine 5-HIAA will be done at SV2. Radiographic scans, computed tomography (CT) or magnetic resonance imaging (MRI) scan will be done during the screening period and evaluated locally by the Investigator to assess tumor stability; radiographic scan will be collected for evaluation by a BICR.

### Eligibility Criteria

### Inclusion Criteria

To be eligible for participation in this study, subjects must meet all the following:

### Gender and Age

1. Male and female subjects aged≥18 years at the time of signing the informed consent form (ICF) prior to initiation of any study specific activities/procedures.

### Type of Subject and Disease Characteristics

2. Confirmed diagnosis of metastatic well-differentiated carcinoid tumor based on most recent histopathology, with associated documented evidence of CS (active diarrhea with or without FEs) anytime in the past, as confirmed by an IAC. The IAC confirmation will be based on medical history, to exclude conditions (other than CS) or treatments that may cause diarrhea.

3. Abnormal plasma or 24-hour urine 5-HIAA assessed at SV1 or SV2 (or within the last 6 months prior to SV1, if both assessments during screening were normal, and following the most recent tumor directed procedure, (e.g., tumor de-bulking, radiolabeled somatostatin analogs [SSA] therapy, chemoembolization), confirmed with the Sponsor on a case-by-case basis).

4. Stable/non progressive tumor as determined by the Investigator.

5. Ki-67 (MIB1 antibody)<20% (or a mitotic index of ≤20 mitoses per 10 high-power fields (2 mm2), if the Ki-67 index could not be quantified reliably).

6. Received parenteral SRL monotherapy, Sandostatin (octreotide) 30 mg or 20 mg or Somatuline (lanreotide) 120 mg, with a stable dose for at least 3 months of therapy at a dosing interval of 4 weeks.

7. Stable BM (average<4.0 BMs/day) during the ˜4 weeks of the SRL injection interval, prior to baseline, as assessed during screening (not including the last day prior to randomization to allow timely calculation and assessment).

### ½Contraception

8. Women of childbearing potential (WOCBP) and men who are sexually active with WOCBP must agree to follow instructions for method(s) of contraception for the duration of the treatment with IP and 30 days after last dose of IP. Contraception methods should be consistent with local regulations.

9. WOCBP must have a negative serum pregnancy test at screening and a negative urine pregnancy test on Day 1.

10. Females not of childbearing potential must be postmenopausal (defined as cessation of regular menstrual periods for at least 12 months) or surgically sterile.

### Informed Consent

11. Subjects are capable of giving signed informed consent which includes compliance with the requirements and restrictions listed in the ICF and in this protocol.

### Exclusion Criteria

Subjects must be excluded from participating in the study if they meet any of the following:

### Medical Conditions

1. Diarrhea associated with any of the following causes:
a) Short bowel syndrome
b) Pancreatic insufficiency; subjects on stable pancreatic enzymes will be allowed, provided diarrhea is not associated with pancreatic insufficiency
c) Diarrhea is only related to SRLs treatment and not to CS (e.g. diarrhea started after initiation of SRL treatment or occurs after each injection with no evidence of diarrhea associated with CS)
d) Known bacterial overgrowth

2. Evidence of current enteric bacterial or viral infection.

3. Diarrhea that meets the definitions of CTCAE v5 Grade 2 (i.e. increase≥4.0-6.0 BM/day, or limiting instrumental ADL) or higher anytime during screening.

4. FEs that meet the definitions of CTCAE v5 Grade 2 (i.e., moderate symptoms or limiting instrumental ADL) or higher anytime during screening.

5. Unstable CS symptoms (mainly BM and FE) or unstable use of anti-diarrheal or antispasmodic medications during the last 3 months per subject's discretion (monthly fluctuations related to the use of long acting SRLs or sporadic symptomatic exacerbations not related to disease progression are accepted).

Unstable BM is defined as BM frequency changed significantly from one month to the other (e.g., from 1-3 BM/day to >4 BM/day, on average).

Unstable FEs, is defined as the frequency and/or severity of FEs changed significantly from one month to the other (e.g., from 1 FE/week to 1 FE/day or from FEs that do not affect daily activities or quality of life to FEs that do affect daily activities or quality of life.

6. Symptomatic abnormalities of the biliary tract.

7. History of unstable angina or acute myocardial infarction within the 12 weeks preceding SV1 or other clinically significant cardiac disease at the time of screening as judged by the Investigator.

8. Any clinically significant uncontrolled nervous system, GI, renal, pulmonary, hepatic concomitant disease, or complications or severe symptoms related to carcinoid tumors (e.g., carcinoid heart disease, or subject unable to tolerate oral medications due to GI complications), that in the Investigator's opinion would preclude subject participation.

9. Uncontrolled diabetes defined as having a fasting glucose>150 mg/dl (8.3 mmol/L) or glycosylated hemoglobin (HbAlc)≥8% (patients can be rescreened after diabetes is brought under adequate control, or in case HbAlc<8%). Subjects with controlled diabetes are allowed (insulin is allowed).

10. Known hypothyroidism or hypocortisolism not adequately treated with a stable dose of thyroid or steroid hormone replacement therapy for ≥12 weeks.

11. Known active Hepatitis B (defined as Hepatitis B surface antigen [HbsAg] reactive) or known active Hepatitis C virus (defined as HCV RNA [qualitative] is detected) infection. Note: No testing for Hepatitis B and Hepatitis C is required unless mandated by local health authority.

12. A history or current evidence of any condition, therapy, or laboratory abnormality that might confound the results of the study, interfere with subject's participation for the full duration of the study, or is not in the best interest of the subject to participate, in the opinion of the treating Investigator.

13. Known history of illicit drug or alcohol abuse within 2 years of screening.

14. Known malignancy, other than metastatic carcinoid tumors, that is progressing or has required active treatment within the past 3 years.

Note: Subjects with basal cell carcinoma of the skin, squamous cell carcinoma of the skin, or carcinoma in situ (e.g., breast carcinoma, cervical cancer in situ) that have undergone potentially curative therapy are not excluded.

15. Active infection requiring systemic therapy.

16. Female subjects who are pregnant or breastfeeding.

### Diagnostic Assessments

17. Eastern Cooperative Oncology Group (ECOG) performance status≥3 at screening.

18. Alanine transaminase (ALT), aspartate aminotransferase (AST) or alkaline phosphatase (ALP)>3 times upper limit of normal (ULN) at screening.

19. Total bilirubin≥1.5×ULN at screening.

20. Estimated glomerular filtration rate (eGFR)<45 mL/min/1.73 m2 calculated by chronic kidney disease epidemiology collaboration (CKD-EPI).

21. Bradycardia (heart rate<50 bpm) or Mean QT interval corrected for heart rate using Fridericia's formula (QTcF)≥450 msec for men or >470 ms for women at screening.

### Prior Therapy

22. Use of telotristat within 4 weeks prior to screening.

23. Use of SC Sandostatin IR for more than 7 out of 28 days during the screening period.

24. Treatment with interferon-alpha, chemotherapy and/or surgical tumor de-bulking within the last 3 months Prior to study entry (SV1).

25. Treatment with selective internal radiation (SIR) therapy (e.g., SIR Spheres) or peptide receptor radionuclide therapy (PRRT) within the last 6 months prior to study entry (SV1).

26. Hepatic arterial embolization or hepatic arterial chemoembolization within the last 3 months prior to study entry (SV1).

27. Undergone major surgery/surgical therapy for any cause within four weeks prior to enrollment or planned procedure during the study.

28. Expected to receive additional medical or surgical treatment for CS during the study.

### Prior/Concurrent Clinical Study Experience

29. Currently participating in or have participated in a study of an investigational agent or has used an investigational device within 4 weeks prior to the first dose of investigational product (IP).

Note: Subjects participating in an observational study are an exception to this criterion and may qualify for the study with Sponsor approval
Note: Subjects who have entered the follow-up phase of an investigational study may participate as long as it has been 4 weeks after the last dose of the previous investigational agent

### Other Exclusions

Known allergy or hypersensitivity to any of the investigational product (IP) or materials.

### PK Assessment

Single-point PK assessments will be collected 3 times (twice during screening and once at baseline, predose). This will allow characterization of the SRL blood levels in the target population of subjects with CS associated with metastatic carcinoid tumors, at different timepoints following the SRL injection.

Twelve-hour PK collection will occur 12 and 20 weeks after DPC Baseline visit to characterize the PK of OOC in this target population in multiple timepoints.

For example, 12-hour PK collection (at the following blood sampling times: predose (within 15 minutes) and at 20, 40, 60, 80, 100, 120, 140, 160 minutes and 3, 3.5, 4, 6, 8, 10 and 12 hours post morning dose) will occur at 12 weeks post DPC baseline visit, and at 20 weeks post DPC baseline visit for subjects enrolled in OLE.

In addition, a 12-hour PK will be collected at baseline of the OLE in subjects meeting the TFC, or who discontinued IP due to treatment failure (not meeting TFC) and were determined to be eligible by the Sponsor's Medical Monitor (or designee) for early roll-over into the OLE and in subjects whose early termination, during the OLE, occurred less than 20 weeks post DPC baseline, to assess the OOC PK in subjects with active disease, e.g., active diarrhea.

### Biomarkers

Plasma and 24-hour urine samples will be collected to assess levels of 5-HIAA.

### Statistical Methods

The primary analysis of the primary estimand/endpoint will be based on the full analysis set, defined as all randomized subjects. The primary analysis will use a stratified Cochran-Mantel-Haenszel (CMH) test, with strata defined by the randomization strata. The primary analysis of the secondary endpoint will use the same approach as the primary endpoint.

To adjust for multiplicity among the primary and secondary endpoints a fixed order of testing will occur. The secondary endpoints will only be tested if the primary endpoint is statistically significant at the 2-sided 5% significance level. Among the secondary endpoints, the second secondary endpoint will only be tested if the first secondary endpoint is statistically significant at the 2-sided 5% significance level.

### Investigational Product

Oral octreotide capsules: OOC (each capsule strength is 20 mg formulated with transient permeability enhancer (TPE) excipients) should be administered as 3 capsules (60 mg) twice per day (BID) (approximately 8 to 12 hours apart) at approximately the same time each day, on an empty stomach, i.e. at least 1 hour prior to a meal or at least 2 hours after a meal. On PK sampling days, subjects will be requested to withhold evening dose until after the 12-hour PK timepoint is collected.

Placebo: Matching placebo capsules, identical in appearance to OOC, containing TPE excipients with no octreotide, should be administered as 3 capsules BID.

### Double-Blind Placebo Controlled (DPC) Treatment Period

The Baseline visit (first IP dose) should be scheduled within +3 days of the intended routine dosing interval following the last SRL injection.

On Day 1/Baseline, eligible subjects will be randomized in a 1:1 ratio to receive 1 of the following treatments for up to 16 weeks or until subjects meet the treatment failure criteria (TFC):
OOC 60 mg BID (3×20 mg capsules); total daily dose 120 mg.
Placebo (3 capsules)

The study will utilize centralized stratified randomization, based on average bowel movement (BM)/day at Baseline (during the last 4 weeks prior to randomization) (<3.0 BM/day vs≥3.0 BM/day) and average flushing episode (FE)/day at Baseline (during the 4 weeks prior to randomization) (<1.0 FE/day vs≥1 FE/day).

The first dose of Investigational Product (IP) will be administered at the clinic. After initiation of IP on Day 1 (Baseline), subjects will return to the clinic and will be evaluated as specified. Assessments will include, but not limited to, collection of daily eDiary (for BMs, and FEs as well as use of anti-diarrheals and/or prohibited Sandostatin SC), patient reported outcomes (PROs), 5-HIAA, and radiographic scans (assessed by BICR).

Use of Sandostatin SC or telotristat or any other systemic treatment for NET or CS will not be allowed during the DPC period. Stable use (compared to the dose and number of days used during the last 4 weeks prior to randomization) of anti-diarrheal and antispasmodic medications will be allowed during the DPC period and should be reported in the eDiary.

### Supplemental Therapy

In case of symptomatic exacerbation of CS, based on recommendation below, supplemental therapy of an additional 40 mg/day (that is, 1 additional 20 mg capsule BID) to a total of 4 capsules BID (equivalent to OOC 80 mg BID, i.e., 160 mg/day) will be allowed in case of symptomatic exacerbation assessed as related to CS, by the investigator. To maintain the blind, subjects on matching placebo will also receive mock supplemental therapy during symptomatic exacerbation of CS symptoms.

Criteria for supplemental therapy:
Increased daily frequency of BM by at least 2 BMs/day, compared to baseline, to ≥4 BMs/day
   or
Increased frequency of FE by at least 1.0 FE/day, compared to baseline, or increased severity of FE (as reported by the eDiary or based on CTCAE v5 grading, respectively).

### Or

New or worsening of other CS signs/symptoms, determined as clinically significant and reported as an adverse event (AE).

Upon symptomatic control, discontinuation of supplemental therapy and return to 3 capsules BID is recommended. Supplemental therapy should not exceed 1 week.

### Dose De-escalation

The dose can be temporarily de-escalated to 2 capsules BID (equivalent to OOC 40 mg BID) on a case-by-case basis, following consultation with the Sponsor, in case of tolerability issues (e.g., severe nausea or headache assessed as related to IP).

### DPC Treatment Failure Criteria

TFC is defined as follows:

Subjects can meet either of the below criteria (i.e., 1 or 2):

1. Increased frequency of BM meeting all of the below criteria (i.e., a, b, and c)
a. Increased BM (any of the below, i through iv)
   i. Average BMs increase by ≥2.0/day compared to baseline and ≥4.0/day over the last 14 days
   ii. Grade 2 diarrhea based on CTCAE v5 criteria (i.e. increase≥4.0-6.0 BM compared to baseline, or limiting instrumental ADL) for at least 7 consecutive days
   iii. Grade 3 diarrhea based on CTCAE v5 criteria (i.e., ≥7.0 BM compared to baseline, or limiting self-care ADL)
   iv. Grade 4 diarrhea based on CTCAE v5 criteria (i.e. life threatening or urgent intervention indicated)
b. Subject received supplementation for intermittent exacerbation for at least 4 of the last 14 days with IP 4 capsules BID (equivalent to OOC dose level of 80 mg/BID in the active arm), except for iii or iv above where this is not required.
c. Increased BM is not related to GI infection, systemic antibiotics or other non-CS etiologies.

2. Worsening FEs, compared to screening period, meeting all the below criteria (i.e., a, b, and c)
a. Worsening FEs (i or ii below)
   i. Grade 2 FEs based on CTCAE v5 criteria (i.e., moderate symptoms, limiting instrumental ADL) for at least 7 of the last 14 days.
   ii. Grade 3 FEs based on CTCAE v5 criteria (i.e., associated with hypotension and/or tachycardia or limiting self-care ADL).
b. Subject received supplementation for intermittent exacerbation for at least 4 of the last 14 days with IP 4 capsules BID (equivalent to OOC dose level of 80 mg/BID in the active arm).
c. Worsening of FEs is not related to a change in diet and alcohol.

### Endpoints

### Primary Endpoint

The study primary endpoint is proportion of responders at the end of the DPC period. Response is defined as average BM<4.0/day during the last 4 weeks of the DPC or average BM/day during the last 4 weeks of the DPC not increased by more than 1.0 BM/day compared to average baseline BM/day, where baseline is based on the average of the last 4 weeks prior to randomization.

### Secondary Endpoints

The secondary endpoints for this study look at proportion of subjects who met TFC related to BM or FE during the DPC.

The 1^{st} secondary endpoint, defined as proportion of subjects who met the TFC related to BM.

The 2^{nd} secondary endpoint, defined as proportion of subjects who met the TFC related to FE during the DP.

### Other Endpoints

This study will look at proportion of subjects who have lost response throughout the DPC period, based on FE, defined as meeting any of the below criteria:
TFC for FE has been met
FE frequency was at least doubled, from screening (last 4 weeks prior to randomization) to the last 4 weeks of treatment to >1 FE/day

This study will look at the change from DPC baseline (last 4 weeks prior to randomization) to end of treatment during the DPC (last 4 weeks on IP during the DPC) in:
Average BMs/day
Average FEs/day

This study will look at change from DPC baseline (defined as assessment at baseline visit) to end of treatment during the DPC in:
Urinary and plasma 5-HIAA
PROs (Carcinoid Syndrome Treatment Satisfaction Questionnaire [CS-TSQ] and Functional Assessment of Cancer Therapy Carcinoid Syndrome Symptom Index [FACT-CSI])

This study will look at change from end of DPC (last 2 weeks on IP during the DPC) to last 2 weeks in OLE (up to OLE Week 12), in average BMs/day

This study will look at change from end of DPC (last 2 weeks on IP during the DPC) to last 2 weeks in OLE (up to OLE Week 12), in average FEs/day

This study will look at change from end of DPC (last assessment) to last available measurement (up to OLE Week 12) in urinary and plasma 5-HIAA

This study will look at change from end of DPC (last assessment) to last available measurement (up to OLE Week 12) in PROs:
CS-TSQ
FACT-CSI

This study will look at PFS defined as the time from initiation of OOC until the date of assessment of objective tumor progression (>20% growth for measurable disease at baseline; becoming measurable for unmeasurable disease at baseline) or death by any cause

This study will look at objective tumor control rate (complete response, partial response or stable disease.

This study will look at change from end of DPC (last 2 weeks on IP during the DPC) to end (last 2 weeks) of each year of the OLE (e.g., prior to OLE Week 48 for the first OLE year, prior to OLE Week 96 for the second year), in subjects responding to OOC during DPC in:
Average BMs/day
Average FEs/day

This study will look at change from end of DPC (last assessment) to end (last assessment on treatment) of each year of the OLE (e.g., prior to OLE Week 48 for the first OLE year, prior to OLE Week 96 for the second year), in subjects responding to OOC during DPC in:
Urinary and plasma 5-HIAA
PROs (CS-TSQ and FACT-CSI).

### Efficacy Assessment

### Clinical Assessments of CS Symptoms

Self-completed daily eDiary will be used to collect data on BMs and FEs as well as use of anti-diarrheals or antispasmodic medications and/or Sandostatin SC (prohibited during DPC) during DPC and OLE at specified timepoints.

### Patient Reported Outcomes (PROs)

### Carcinoid Syndrome—Treatment Satisfaction Questionnaire (CS-TSQ)

A treatment satisfaction questionnaire for adult subjects with CS symptoms will be used in this study and is intended to be completed by the subject (i.e. PRO). This is a modified version of the Acromegaly (ACRO)-TSQ which was developed by Chiasma (acquired by Amryt) according to best practices for PRO development and was validated in 79 acromegaly patients.

The CS-TSQ is focused on the following aspects:
CS Symptoms
Injection Site Reactions
Treatment Administration Bother
Treatment Convenience
Emotional Impact
Treatment Satisfaction

### Patient Perception Questionnaire

To assess subject perception of the observed changes in BM frequency and in the severity/frequency of FE is meaningful for the patients, a Patient Perception Questionnaire (PPQ) will be utilized as an anchor-based analysis. At Week 16/ET visit, subjects will be asked to complete the Patient Perception Questionnaire to address their perception of change in BM and FE (3 separate questions), on IP (oral) compared to SRL injections (prior to enrollment into the study), (“much better”, “a little better”, “the same”, “a little worse”, “much worse”), and their overall satisfaction with IP (oral) compared to SRL injections in control of their BM and FE (additional 2 questions) (“very satisfied”, “satisfied”, “neither satisfied nor dissatisfied”, “dissatisfied”, “very dissatisfied”).

### Functional Assessment of Cancer Therapy—Carcinoid Syndrome Symptom Index (FACT-CSI)

The FACT-CSI assesses the presence of symptoms like diarrhea and includes associated functional limitations (i.e., embarrassment, urgency, limiting activities, fears of being far from the toilet) (Shaunfield, 2020). The questionnaires contain 20 items. Each item is graded on a 0 to 4 point scale.

### Response Evaluation Criteria in Solid Tumors (RECIST) v1.1 and Radiographic Imaging

Progression free survival and objective tumor response rate will be evaluated using RECIST v1.1 (Eisenhauer, 2009).

Radiographic imaging (CT or MRI) will be collected for evaluation by BICR at timepoints specified in SoA (Section 1.3.1—DPC and Section 1.3.2—OLE). During the OLE, radiographic imaging (CT or MRI) will be performed every 3 months.

### Safety Assessments

Safety will be evaluated throughout with AE monitoring and other safety assessments.

### Open-label Extension Study

### Eligibility Criteria for Open-label Extension

1. Completed the full duration of the DPC per protocol or subject who met the TFC during the DPC, or subject who discontinued IP due to treatment failure (without meeting TFC) and determined by the Sponsor's Medical Monitor (or designee) to be eligible to early roll-over into the OLE. Such a decision will be based on the eDiary information, a clinical narrative provided by the Investigator, and Sponsor's Medical Monitor (or designee) determination that the noted symptomatic exacerbation is related to CS and is clinically meaningful.

2. For subjects completing the full duration of the DPC, carcinoid tumor and CS symptoms are adequately controlled per Investigator's discretion.

3. No tumor progression based on radiographic imaging at the end of the DPC (imaging within the last month prior to DPC Week 16 is acceptable).

4. IP is not withheld for an IP-related TEAE.

5. For subjects who discontinue IP during the DPC due to Grade 3 diarrhea, requiring hospitalization or Grade 4 diarrhea, resolution of these TEAEs is required.

6. No clinically significant or unstable medical or surgical condition detected or worsened during the study, which would preclude safe participation and completion of the OLE.

7. OOC are not commercially available for the treatment of CS in the applicable region or country.

8. Willing and able to comply with the protocol requirements for the duration of the OLE.

9. Agree to continue treatment with OOC and able to understand and sign an additional written informed consent prior to entering the OLE

Planned Sample Size A total of up to 100 subjects are planned.

### Treatment

Beginning at the OLE Enrollment visit (same as the last visit of DPC), all subjects will receive OOC 60 mg BID until they meet the TFC in the OLE the last subject enrolled into the OLE completes 96 weeks or until IP marketing for this indication or study termination.

Use of Sandostatin SC or telotristat or any other systemic treatment for NET or CS will not be allowed during the OLE. Stable use (compared to the dose and number of days used during the last 4 weeks prior to randomization) of anti-diarrheal and antispasmodic medications will be allowed during the OLE period and should be reported in the eDiary. Supplemental therapy for CS symptomatic exacerbation will be allowed as described above.

During the initial 12 weeks of the OLE, daily eDiary of BMs and FEs will be collected as well as use of anti-diarrheals and/or prohibited Sandostatin SC. Following OLE Week 12, eDiary will be collected only 2 weeks prior to each in-clinic visit. PROs will be assessed (CS-TSQ and FACT-CSI) at OLE Week 12, Week 48 and Week 96. Radiographic imaging (CT/MRI) will be performed every 3 months; radiographic scans will be evaluated by BICR.

1. A method for treating diarrhea and flushing episodes associated with carcinoid tumors in a patient in need thereof, comprising orally administering a total daily dose of at least about 120 mg of octreotide or a pharmaceutically acceptable salt thereof to the patient.

2. The method of claim 1, wherein octreotide is administered for the long-term maintenance treatment of diarrhea and flushing episodes associated with a carcinoid tumor.

3. The method of claim 1, wherein the carcinoid tumor is a metastatic carcinoid tumor.

4. The method of claim 1, wherein octreotide is administered for at least about 2 weeks, 4 weeks, 12 weeks, 16 weeks, 20 weeks, 26 weeks, 40 weeks, 1 year or 2 years.

5. The method of claim 1, wherein about 120 mg of octreotide is administered per day.

6. The method of claim 1, wherein about 160 mg of octreotide is administered per day.

7. The method of claim 5, further comprising:
determining the patient's daily frequency of bowel movements (BMs) following administration of 120 mg of octreotide per day; and
administering about 160 mg of octreotide per day to the patient if:
(a) the patient experiences an increased daily frequency of BMs by at least 2 BMs, compared to baseline prior to the administration of 120 mg of octreotide; and
(b) the patient experiences≥4 BMs/day following administration of 120 mg of octreotide.

8. The method of claim 5, further comprising,
determining the patient's daily frequency of flushing episode (FE) following administration of 120 mg of octreotide per day, and
administering about 160 mg of octreotide per day to the patient if:
(a) the patient experiences an increased daily frequency of FEs by at least 1 FE per day compared to prior to the administration of 120 mg of octreotide; or
(b) the patient experiences an increased severity of FE following administration of 120 mg of octreotide.

9. The method of claim 8, wherein the patient has increased severity of FE as determined by an eDiary or based on CTCAE v5 grading.

10. The method of claim 5, further comprising:
determining the patient's carcinoid syndrome signs/symptoms following administration of 120 mg of octreotide per day; and
administering about 160 mg of octreotide per day to the patient if the patient experiences new or worsening carcinoid syndrome signs/symptoms.

11. The method of claim 1, wherein 160 mg of octreotide is administered per day for about 4 days to about 7 days.

12. The method of claim 1, further comprising:
determining the patient's carcinoid syndrome signs/symptoms following administration of 160 mg of octreotide per day; and
administering about 120 mg of octreotide per day to the patient if the patient's biochemical and/or symptomatic response is controlled.

13. The method of claim 5, further comprising:
determining the patient's tolerability following administration of 120 mg of octreotide per day; and
administering about 80 mg of octreotide per day to the patient if the patient experiences tolerability issues following administration of 120 mg of octreotide per day.

14. The method of claim 1, wherein octreotide is administered twice per day.

15. The method of claim 14, wherein the two octreotide doses are administered at least about 8 to about 12 hours apart.

16. The method of claim 14, wherein octreotide is administered in the morning and in the evening.

17. The method of claim 1, wherein octreotide is administered in capsules.

18. The method of claim 17, wherein each capsule contains 20 mg of octreotide.

19. The method of claim 1, wherein the patient treated with oral octreotide has had prior treatment with a somatostatin receptor ligand (SRL) that has been shown to be effective and tolerated.

20. The method of claim 19, wherein the SRL is an injectable SRL.

21. The method of claim 20, wherein the injectable SRL is octreotide, lanreotide or pasireotide.

22. The method of claim 21, wherein the injectable SRL is octreotide or lanreotide.

23. The method of claim 22, wherein the injectable octreotide is a long-acting release (LAR) octreotide formulation or a subcutaneous (SC) immediate release (IR) octreotide formulation.

24. The method of claim 1, wherein the patient was administered 10 mg, 20 mg or 30 mg of octreotide or 120 mg of lanreotide in the prior treatment.

25. The method of claim 1, wherein the administering of octreotide or a pharmaceutically acceptable salt thereof occurs at least 1 hour before a meal or at least 2 hours after a meal.

26. The method of claim 1, wherein the administering of octreotide or a pharmaceutically acceptable salt thereof occurs on an empty stomach.

27. The method of claim 1, wherein octreotide is administered in a capsule comprising an oily suspension.

28. The method of claim 27, wherein the oily suspension comprises an admixture of a hydrophobic medium and a solid form, wherein the solid form comprises octreotide and at least one salt of a medium chain fatty acid.

29. The method of claim 27, wherein the oily suspension comprises an admixture of a hydrophobic medium and a solid form, wherein the solid form comprises octreotide, at least one salt of a medium chain fatty acid, and polyvinylpyrrolidone (PVP), wherein the at least one salt of a medium chain fatty acid is present in the dosage form at an amount of at least 12% by weight and wherein the PVP is present in the dosage form at an amount of 3% or more by weight.

30. The method of claim 27, wherein the composition comprises octreotide, about 12-21% of sodium octanoate by weight, about 5-15% of polyvinylpyrrolidone by weight, about 20-80% of glyceryl tricaprylate by weight, and about 3-10% of surfactant by weight.

31. The method of claim 17, wherein the capsule is enterically coated.

32. The method of claim 1, wherein octreotide comprises octreotide acetate.

33. The method of claim 1, wherein, following administration of the oral octreotide, the patient experiences an improvement in diarrhea and flushing episodes associated with carcinoid tumors.

34. The method of claim 1, wherein, following administration of the oral octreotide, the patient has an average number of daily bowl movements of less than about 4.

35. The method of claim 4, wherein after about 16 weeks of treatment the patient has an average number of daily bowel movements of less than about 4 during the last 4 weeks (i.e. week 13-16).

36. The method of claim 1, wherein after said treating the patient has an average number of bowel movements per day (BM/day) that has not increased by more than 1.0 BM/day compared to baseline prior to treatment.

37. The method of claim 1, wherein after said treating the patient has tolerable FEs if present, defined as Grade 1, Grade 2, or Grade 3 by CTCAE v5 criteria.

38. A method of treating carcinoid syndrome in a patient in need thereof comprising orally administering a total daily dose of at least about 120 mg of octreotide or a pharmaceutically acceptable salt thereof to the patient.

39. The method of claim 38, wherein octreotide is administered for at least about 2 weeks, 4 weeks, 12 weeks, 16 weeks, 20 weeks, 26 weeks, 40 weeks, 1 year or 2 years.

40. The method of claim 38, wherein about 120 mg of octreotide is administered per day.

41. The method of claim 38, wherein about 160 mg of octreotide is administered per day.

42. The method of claim 40, further comprising:
determining the patient's daily frequency of bowel movements (BMs) following administration of 120 mg of octreotide per day; and
administering about 160 mg of octreotide per day to the patient if:
(a) the patient experiences an increased daily frequency of BMs by at least 2 BMs, compared to baseline prior to the administration of 120 mg of octreotide; and
(b) the patient experiences≥4 BMs/day following administration of 120 mg of octreotide.

43. The method of claim 40, further comprising,
determining the patient's daily frequency of flushing episode (FE) following administration of 120 mg of octreotide per day, and
administering about 160 mg of octreotide per day to the patient if:
(a) the patient experiences an increased daily frequency of FEs by at least 1 FE per day compared to prior to the administration of 120 mg of octreotide; or
(b) the patient experiences an increased severity of FE following administration of 120 mg of octreotide.

44. The method of claim 41, wherein the patient has increased severity of FE as determined by an eDiary or based on CTCAE v5 grading.

45. The method of claim 40, further comprising:
determining the patient's carcinoid syndrome signs/symptoms following administration of 120 mg of octreotide per day; and
administering about 160 mg of octreotide per day to the patient if the patient experiences new or worsening carcinoid syndrome signs/symptoms.

46. The method of claim 41, wherein 160 mg of octreotide is administered per day for about 4 days to about 7 days.

47. The method of claim 41, further comprising:
determining the patient's carcinoid syndrome signs/symptoms following administration of 160 mg of octreotide per day; and
administering about 120 mg of octreotide per day to the patient if the patient's biochemical and/or symptomatic response is controlled.

48. The method of claim 40, further comprising:
determining the patient's tolerability following administration of 120 mg of octreotide per day; and
administering about 80 mg of octreotide per day to the patient if the patient experiences tolerability issues following administration of 120 mg of octreotide per day.

49. The method of claim 38, wherein octreotide is administered twice per day.

50. The method of claim 49, wherein the two octreotide doses are administered at least about 8 to about 12 hours apart.

51. The method of claim 49, wherein octreotide is administered in the morning and in the evening.

52. The method of claim 38, wherein octreotide is administered in capsules.

53. The method of claim 52, wherein each capsule contains 20 mg of octreotide.

54. The method of claim 38, wherein the patient treated with oral octreotide has had prior treatment with a somatostatin receptor ligand (SRL) that has been shown to be effective and tolerated.

55. The method of claim 54, wherein the SRL is an injectable SRL.

56. The method of claim 55, wherein the injectable SRL is octreotide, lanreotide or pasireotide.

57. The method of claim 56, wherein the injectable SRL is octreotide or lanreotide.

58. The method of claim 57, wherein the injectable octreotide is a long-acting release (LAR) octreotide formulation or a subcutaneous (SC) immediate release (IR) octreotide formulation.

59. The method of claim 56, wherein the patient was administered 10 mg, 20 mg or 30 mg of octreotide or 120 mg of lanreotide in the prior treatment.

60. The method of claim 38, wherein the administering of octreotide or a pharmaceutically acceptable salt thereof occurs at least 1 hour before a meal or at least 2 hours after a meal.

61. The method of claim 38, wherein the administering of octreotide or a pharmaceutically acceptable salt thereof occurs on an empty stomach.

62. The method of claim 38, wherein octreotide is administered in a capsule comprising an oily suspension.

63. The method of claim 62, wherein the oily suspension comprises an admixture of a hydrophobic medium and a solid form, wherein the solid form comprises octreotide and at least one salt of a medium chain fatty acid.

64. The method of claim 62, wherein the oily suspension comprises an admixture of a hydrophobic medium and a solid form, wherein the solid form comprises octreotide, at least one salt of a medium chain fatty acid, and polyvinylpyrrolidone (PVP), wherein the at least one salt of a medium chain fatty acid is present in the dosage form at an amount of at least 12% by weight and wherein the PVP is present in the dosage form at an amount of 3% or more by weight.

65. The method of claim 62, wherein the composition comprises octreotide, about 12-21% of sodium octanoate by weight, about 5-15% of polyvinylpyrrolidone by weight, about 20-80% of glyceryl tricaprylate by weight, and about 3-10% of surfactant by weight.

66. The method of claim 52, wherein the capsule is enterically coated.

67. The method of claim 38, wherein octreotide comprises octreotide acetate.

68. The method of claim 38, wherein, following administration of the oral octreotide, the patient experiences an improvement in diarrhea and/or flushing episodes associated with carcinoid syndrome.

69. The method of claim 38, wherein, following administration of the oral octreotide, the patient has an average number of daily bowl movements of less than about 4.

70. The method of claim 38, wherein after about 16 weeks of treatment the patient has an average number of daily bowl movements of less than about 4 during the last 4 weeks (i.e. week 13-16).

71. The method of claim 38, wherein after said treating the patient has an average number of bowel movements per day (BM/day) that has not increased by more than 1.0 BM/day compared to baseline prior to treatment.

72. The method of claim 39, wherein after said treating the patient has tolerable FEs if present, defined as Grade 1, Grade 2, or Grade 3 by CTCAE v5 criteria.
